(19) United States
(12) Patent Application Publication
(10) Pub. No.: US 2024/0002779 A1
(43) Pub. Date: Jan. 4, 2024

(54) Functionalization of Mycelium Materials

(71) Applicant: BOLT THREADS, INC., Emeryville, CA (US)

(72) Inventors: Matthew Jordan Smith, Emeryville, CA (US); Namrata Vinay Patil, Emeryville, CA (US); Matthew Howard Farmer, Emeryville, CA (US)

(21) Appl. No.: 18/254,129

(22) PCT Filed: Nov. 24, 2021
(86) PCT No.: PCT/US21/60737

§ 371 Date: May 23, 2023

Related U.S. Application Data
(60) Provisional application No. 63/147,620, filed on Feb. 9, 2021.
(60) Provisional application No. 63/117,897, filed on Nov. 24, 2020.

(51) Int. Cl. C12N 1/14

(52) U.S. Cl. CPC C12N 1/14

(57) ABSTRACT

Provided herein are mycelium materials and methods for production thereof. In some embodiments, a mycelium material includes: a cultivated mycelium material including one or more masses of branching hyphae, wherein the one or more masses of branching hyphae may be disrupted or pressed and a siloxane or an aliphatic chain compound may be combined with the cultivated mycelium material. Methods of producing a mycelium material are also provided.

### CROSS REFERENCE TO RELATED APPLICATIONS

This application claims the benefit of U.S. Provisional Application No. 63/117,897, filed Nov. 24, 2020, and U.S. Provisional Application No. 63/147,620, filed Feb. 9, 2021, both of which are hereby incorporated in their entirety by reference.

### BACKGROUND

Due to its bioefficiency, strength, and low environmental footprint, mycelium is of increasing interest in the next generation of sustainable materials. However, the mycelium materials currently undergoing development have poor mechanical qualities, including brittleness, susceptibility to delamination and tearing under stress, and non-uniform aesthetic qualities. What is needed, therefore, are improved mycelium materials with favorable mechanical properties, aesthetic properties, and other advantages, as well as materials and methods for making improved mycelium materials.

### SUMMARY

In one aspect, provided herein is a composite mycelium material, comprising a cultivated mycelium material comprising one or more masses of branching hyphae, and an aliphatic chain compound covalently linked to the one or more masses of branching hyphae.

In another aspect, provided herein is a composite mycelium material, comprising a cultivated mycelium material comprising one or more masses of branching hyphae, and a siloxane.

In some embodiments, the one or more masses of branching hyphae is disrupted.

In some embodiments, the cultivated mycelium material is pressed.

In some embodiments, the aliphatic chain compound comprises 2-octenyl succinic anhydride (OSA), 2-dodecenyl succinic anhydride, octadecenyl succinic anhydride, stearic anhydride, 3-Chloro-2-hydroxypropyldimethyldodecylammonium chloride, heptanoic anhydride, butyric anhydride, or a chlorohydrin.

In some embodiments, the siloxane comprises a hydroxysilicone, a silicone hydride, an epoxy silicone, an aminosilicone, or an alkyl ethylene oxide condensate.

In some embodiments, the composite mycelium material comprising an aliphatic chain compound has a lower flexural modulus as compared to a cultivated mycelium material alone.

In some embodiments, the composite mycelium material comprising a siloxane has a lower flexural modulus as compared to a cultivated mycelium material alone.

In some embodiments, the composite mycelium material has a flexural modulus of less than 80 MPa.

In some embodiments, the composite mycelium material has a flexural modulus of 1 MPa to 80 MPa.

In some embodiments, the composite mycelium material has a flexural modulus of at least about 1, 5, 10, 15, 20, 25, 30, 35, 40, 45, 50, 55, 60, 65, 70, 75, or 80 MPa.

In some embodiments, the composite mycelium material is more flexible as compared to a cultivated mycelium material alone.

In some embodiments, the composite mycelium material further comprises a bonding agent.

In some embodiments, the bonding agent comprises one or more reactive groups.

In some embodiments, the one or more reactive groups react with active hydrogen containing groups.

In some embodiments, the active hydrogen containing groups comprise amine, hydroxyl, and carboxyl groups.

In some embodiments, the bonding agent comprises an adhesive, a resin, a crosslinking agent, and/or a matrix.

In some embodiments, the bonding agent is selected from the group consisting of a vinyl acetate-ethylene (VAE) copolymer, a vinyl acetate-acrylic copolymer, a polyamide-epichlorohydrin resin (PAE), a copolymer, transglutaminase, citric acid, genipin, alginate, gum arabic, latex, a natural adhesive, and a synthetic adhesive.

In some embodiments, the bonding agent is a copolymer with a property selected from the group consisting of: a particle size of less than or equal to 1 μm, a sub-zero glass transition temperature, and self-crosslinking function.

In some embodiments, the bonding agent is a vinyl acetate-ethylene (VAE) copolymer.

In some embodiments, the composite mycelium material further comprises a dye.

In some embodiments, the dye is selected from the group consisting of an acid dye, a direct dye, a synthetic dye, a natural dye, and a reactive dye.

In some embodiments, the composite mycelium material is colored with the dye and the color of the composite mycelium material is substantially uniform on one or more surfaces of the composite mycelium material.

In some embodiments, the dye is present throughout the interior of the composite mycelium material.

In some embodiments, the composite mycelium material further comprises a plasticizer.

In some embodiments, the plasticizer is selected from the group consisting of oil, glycerin, fatliquor, sorbitol, diethyloxyester dimethyl ammonium chloride, Tween 20, Tween 80, m-erythritol, water, glycol, triethyl citrate, water, acetylated monoglycerides, and epoxidized soybean oil.

In some embodiments, the composite mycelium material further comprises a tannin.

In some embodiments, the composite mycelium material further comprises a finishing agent.

In some embodiments, the finishing agent is selected from the group consisting of urethane, wax, nitrocellulose, and a plasticizer.

In some embodiments, the cultivated mycelium material has been generated on a solid substrate.

In some embodiments, the one or more masses of branching hyphae are entangled, wherein the entangling the hyphae comprises hydroentangling, needle punching or felting.

In some embodiments, the one or more masses of branching hyphae is disrupted by a mechanical action.

In some embodiments, the mechanical action comprises blending the one or more masses of branching hyphae.

In some embodiments, the mechanical property comprises a wet tensile strength, an initial modulus, an elongation percentage at the break, a thickness, and/or a slit tear strength.

In another aspect, provided herein is a method of producing a composite mycelium material, the method comprising: generating a cultivated mycelium material comprising one or more masses of branching hyphae; and adding a siloxane to the cultivated mycelium material; thus producing the composite mycelium material.

In some embodiments, the method further comprises disrupting or pressing the cultivated mycelium material generated in step (a).

In some embodiments, the siloxane is added before the masses of branching hyphae are disrupted, during disruption of the masses of branching hyphae, or after the disruption of the masses of branching hyphae.

In some embodiments, the siloxane is added before the pressing step, during the pressing step, or after the pressing step.

In some embodiments, the siloxane comprises a hydroxysilicone, a silicone hydride, an epoxy silicone, an aminosilicone, or an alkyl ethylene oxide condensate.

In some embodiments, the cultivated mycelium material comprising a siloxane has a lower flexural modulus as compared to a cultivated mycelium material without a siloxane.

In another aspect, provided herein is a method of producing a composite mycelium material, the method comprising: generating a cultivated mycelium material comprising one or more masses of branching hyphae; and adding an aliphatic chain compound to the cultivated mycelium material; thus producing the composite mycelium material.

In some embodiments, the method further comprises disrupting or pressing the cultivated mycelium material generated in step (a).

In some embodiments, the aliphatic chain compound is added before the masses of branching hyphae are disrupted, during disruption of the masses of branching hyphae, or after the disruption of the masses of branching hyphae.

In some embodiments, the aliphatic chain compound is added before the pressing step, during the pressing step, or after the pressing step.

In some embodiments, the aliphatic chain compound comprises 2-octenyl succinic anhydride (OSA), 2-dodecenyl succinic anhydride, octadecenyl succinic anhydride, stearic anhydride, 3-Chloro-2-hydroxypropyldimethyldodecylammonium chloride, heptanoic anhydride, butyric anhydride, or a chlorohydrin.

In some embodiments, the cultivated mycelium material comprising an aliphatic chain compound has a lower flexural modulus as compared to a cultivated mycelium material without an aliphatic chain compound.

In some embodiments, the composite mycelium material has a flexural modulus of less than 80 MPa.

In some embodiments, the composite mycelium material has a flexural modulus of 1 MPA to 80 MPa.

In some embodiments, the composite mycelium material has a flexural modulus of at least about 1, 5, 10, 15, 20, 25, 30, 35, 40, 45, 50, 55, 60, 65, 70, 75, or 80 MPa.

In some embodiments, the composite mycelium material is more flexible as compared to a cultivated mycelium material alone.

In some embodiments, composite mycelium material further comprises a bonding agent.

In some embodiments, the bonding agent comprises one or more reactive groups.

In some embodiments, the one or more reactive groups react with active hydrogen containing groups.

In some embodiments, the active hydrogen containing groups comprise amine, hydroxyl, and carboxyl groups.

In some embodiments, the bonding agent comprises an adhesive, a resin, a crosslinking agent, and/or a matrix.

In some embodiments, the bonding agent is selected from the group consisting of a vinyl acetate-ethylene (VAE) copolymer, a vinyl acetate-acrylic copolymer, a polyamide-epichlorohydrin resin (PAE), a copolymer, transglutaminase, citric acid, genipin, alginate, gum arabic, latex, a natural adhesive, and a synthetic adhesive.

In some embodiments, the bonding agent is a copolymer with a property selected from the group consisting of: a particle size of less than or equal to 1 μm, a sub-zero glass transition temperature, and self-crosslinking function.

In some embodiments, the bonding agent is a vinyl acetate-ethylene (VAE) copolymer.

In some embodiments, the composite mycelium material further comprises a dye.

In some embodiments, the dye is selected from the group consisting of an acid dye, a direct dye, a synthetic dye, a natural dye, and a reactive dye.

In some embodiments, the composite mycelium material is colored with the dye and the color of the composite mycelium material is substantially uniform on one or more surfaces of the composite mycelium material.

In some embodiments, the dye is present throughout the interior of the composite mycelium material.

In some embodiments, the composite mycelium material further comprises a plasticizer.

In some embodiments, the plasticizer is selected from the group consisting of oil, glycerin, fatliquor, sorbitol, diethyloxyester dimethyl ammonium chloride, Tween 20, Tween 80, m-erythritol, water, glycol, triethyl citrate, water, acetylated monoglycerides, and epoxidized soybean oil.

In some embodiments, the composite mycelium material further comprises a tannin.

In some embodiments, the composite mycelium material further comprises a finishing agent.

In some embodiments, the finishing agent is selected from the group consisting of urethane, wax, nitrocellulose, and a plasticizer.

In some embodiments, the cultivated mycelium material has been generated on a solid substrate.

In some embodiments, the method further comprises entangling the one or more masses of branching hyphae, wherein the entangling the hyphae comprises hydroentangling, needle punching, or felting.

In some embodiments, the disrupting comprises disrupting the one or more masses of branching hyphae by a mechanical action.

In some embodiments, the mechanical action comprises blending the one or more masses of branching hyphae.

### BRIEF DESCRIPTION OF THE FIGURES

FIG. 1 depicts a schematic diagram of methods of producing a composite mycelium material according to some embodiments described herein. A box having a solid line indicates a required step and a box having a dashed line indicates an optional step.

FIG. 2 shows a flowchart of a method of producing a material comprising mycelium and a siloxane or aliphatic chain compound.

FIG. 3 shows the incorporation of the OSA into the treated material as determined by ATR-FTIR.

FIG. 4 shows the slit tear test results for the indicated materials.

FIG. 5 shows the T-peel tear test results for the indicated materials.

FIG. 6A shows the flexural modulus test results for the control mycelium material.

FIG. 6B shows the flexural modulus test results for the OSA only treated mycelium material.

FIG. 6C shows the flexural modulus test results for the OSA+5 g Elite Plus binder treated mycelium material. FIG. 6D shows the flexural modulus test results for the OSA+9.8 g Elite Plus binder treated mycelium material.

### DETAILED DESCRIPTION

### Definitions

The details of various embodiments of the disclosure are set forth in the description below. Other features, objects, and advantages of the disclosure will be apparent from the description. Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The following terms, unless otherwise indicated, shall be understood to have the following meanings:

The term “hyphae” refers to a morphological structure of a fungus that is characterized by a branching filamentous shape.

The term “hyphal” refers to an object having a component thereof comprised of hyphae.

The term “mycelium” refers to a structure formed by one or more masses of branching hyphae. A “mass” refers to a quantity of matter. Mycelium is a distinct and separate structure from a fruiting body of a fungus or sporocarp.

The terms “cultivate” and “cultivated” refer to the use of defined techniques to deliberately grow a fungus or other organism.

The term “cultivated mycelium material” refers to material that includes one or more masses of cultivated mycelium, or includes solely of cultivated mycelium. In some embodiments, the one or more masses of cultivated mycelium is disrupted as described herein. In most cases, the cultivated mycelium material has been generated on a solid substrate, as described below.

The term “composite mycelium material” refers to any material including cultivated mycelium material combined with another material, such as a lubricant as described herein. Lubricants include, but are not limited to, a siloxane or an aliphatic chain compound described herein.

In some embodiments, the mycelium comprises a supporting material. Suitable supporting materials include, but are not limited to, a mass of contiguous, disordered fibers (e.g. non-woven fibers), a perforated material (e.g. metal mesh, perforated plastic), a mass of discontiguous particles (e.g. pieces of woodchip) or any combination thereof. In specific embodiments, the supporting material is selected from the group consisting of a mesh, a cheesecloth, a fabric, a knit, a woven, and a non-woven textile. In some embodiments, the mycelium comprises a reinforcing material. A reinforcing material is a supporting material that is entangled within a mycelium or composite mycelium material. In some embodiments, the mycelium comprises a base material. A base material is a supporting material that is positioned on one or more surfaces of the mycelium or composite mycelium material.

The term “incorporate” refers to any substance, e.g., cultivated mycelium material, composite mycelium material, or a lubricant, that can be combined with or contacted with another substance. In a specific embodiment, a mycelium or composite mycelium material can be combined with, contacted with, or incorporated into a supporting material, e.g., woven, twisted, wound, folded, entwined, entangled, or braided together, to produce a mycelium material that has become incorporated with the supporting material. In another embodiment, one or more lubricants may be incorporated within the cultivated mycelium material, either in its disrupted or undisrupted state, e.g., embedded throughout the material, or added as a thin coating layer, such as by spraying, saturation, dipping, nip rolling, coating, and the like, to produce a mycelium material.

As used herein, the term “disrupted” with respect to one or more masses of branching hyphae refer to one or more masses of branching hyphae of which one or more disruptions have been applied. A “disruption,” as described herein, may be mechanical or chemical, or a combination thereof. In some embodiments, the one or more masses of branching hyphae is disrupted by a mechanical action. A “mechanical action” as used herein refers to a manipulation of or relating to machinery or tools. Exemplary mechanical actions include, but are not limited to, blending, chopping, impacting, compacting, bounding, shredding, grinding, compressing, high-pressure, shearing, laser cutting, hammer milling, and waterjet forces. In some embodiments, a mechanical action may include applying a physical force, e.g., in one or more directions such that the at least some of the masses of branching hyphae are aligned in parallel in one or more directions, wherein the physical force is applied repeatedly. In some other embodiments, the one or more masses of branching hyphae is disrupted by chemical treatment. “Chemical treatment” as used herein refers to contacting the cultivated mycelium material or composite mycelium material with a chemical agent, e.g., a base or other chemical agent, in an amount sufficient to cause a disruption. In various embodiments, a combination of mechanical actions and chemical treatments may be used herein. The amount of mechanical action (for example, the amount of pressure) and/or chemical agent applied, the period of time for which the mechanical action and/or chemical treatment is applied, and the temperature at which the mechanical action and/or chemical agent is applied, depends, in part, on the components of the cultivated mycelium material or composite mycelium material, and are selected to provide an optimal disruption on the cultivated mycelium material or composite mycelium material.

The term “lubricant” as used herein refers to any molecule that interacts with a structure to increase mobility of the structure.

The term “processed mycelium material” as used herein refers to a mycelium that has been post-processed by any combination of treatments with preserving agents, plasticizers, finishing agents, dyes, and/or protein treatments.

The term “web” as used herein refers to a mycelium material or composite mycelium material that has been disrupted, converted into a slurry, and arranged in a formation (e.g. drylaid, airlaid and/or wetlaid).

The term “spunlace” as used herein refers to a mycelium material or composite mycelium material that has been disrupted and hydroentangled, wherein one or more masses of branching hyphae are entangled using jets of water or the like.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the aspects of the present disclosure. The upper and lower limits of these smaller ranges may independently be included in the smaller ranges, and are also encompassed within the aspects of the present disclosure, subject to any specifically excluded limit in the stated range. Where the stated range includes one or both of the limits, ranges excluding either or both of those included limits are also included in the aspects of the present disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Exemplary methods and materials are described below, although methods and materials similar or equivalent to those described herein can also be used in the practice of the present disclosure and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Mycelium Compositions and Methods of Production

Provided herein are cultivated mycelium materials and composite mycelium materials and scalable methods of producing the cultivated mycelium materials and composite mycelium materials. In some or most embodiments, the composite mycelium materials include a cultivated mycelium material having one or more masses of branching hyphae, and a siloxane. In some or most embodiments, the composite mycelium materials include a cultivated mycelium material having one or more masses of branching hyphae, and an aliphatic chain compound. In some or most embodiments, the composite mycelium materials include a cultivated mycelium material having one or more masses of branching hyphae, and a lubricant. In some embodiments, the one or more masses of branching hyphae is disrupted. In some embodiments, the cultivated mycelium material is pressed. Methods of producing the cultivated mycelium material and composite mycelium material are also provided.

Exemplary patents and applications discussing methods of growing mycelium include, but are not limited to: PCT Publication No. 1999/024555; G.B. Patent No. 2,148,959; G.B. Patent No. 2,165,865; U.S. Pat. Nos. 5,854,056; 2,850,841; 3,616,246; 9,485,917; 9,879,219; 9,469,838; 9,914,906; 9,555,395; U.S. Patent Publication No. 2015/0101509; U.S. Patent Publication No. 2015/0033620, all of which are incorporated herein by reference in their entirety. U.S. Patent Publication No. 2018/0282529, published on Oct. 4, 2018 discusses various mechanisms of solution-based post-processing mycelium material to produce a material that has favorable mechanical characteristics for processing into a textile or leather alternative.

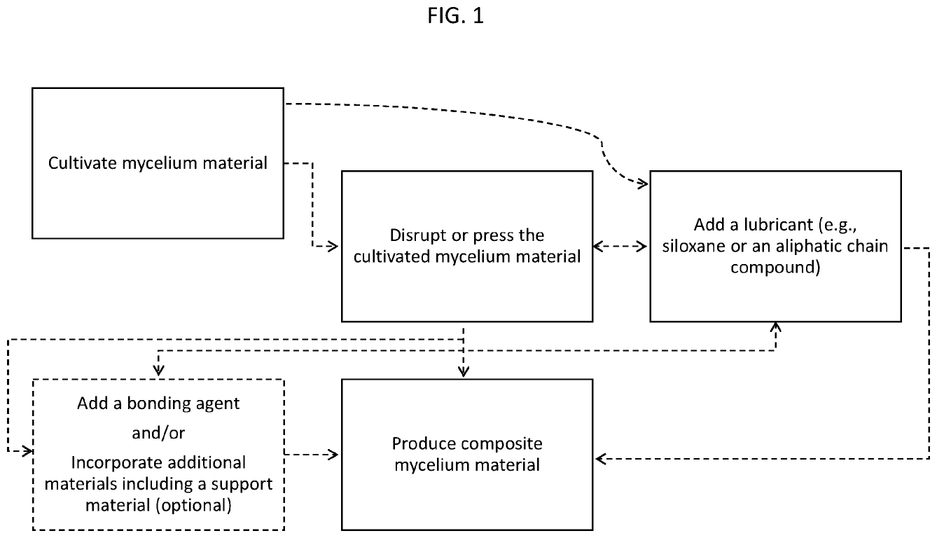

As shown in FIG. 1, exemplary methods of producing mycelium materials according to some embodiments described herein include cultivating mycelium material, optionally disrupting or pressing the cultivated mycelium material, optionally adding a lubricant, such as a siloxane or aliphatic chain compound, optionally incorporating additional materials such as a support material, and combinations thereof. In various embodiments, traditional paper milling equipment may be adapted or used to perform some, or all, of the steps presented herein. In such embodiments, the mycelium material is produced using traditional paper milling equipment.

A description of an embodiment with several components in communication with each other does not imply that all such components are required. To the contrary, a variety of optional components may be described to illustrate a wide variety of possible embodiments of one or more aspects of the present disclosure and in order to more fully illustrate one or more aspects of the present disclosure. Similarly, although process steps, method steps, algorithms or the like may be described in sequential order, such processes, methods, and algorithms may generally be configured to work in alternate orders, unless specifically stated to the contrary. In other words, any sequence or order of steps that may be described herein does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more embodiments, and does not imply that the illustrated process is preferred. Also, steps are generally described once per embodiment, but this does not mean they must occur once, or that they may only occur once each time a process, method, or algorithm is carried out or executed. Some steps may be omitted in some embodiments or some occurrences, or some steps may be executed more than once in a given embodiment or occurrence.

Cultivated Mycelium Material

Embodiments of the present disclosure include various types of cultivated mycelium materials. Depending on the particular embodiment and requirements of the material sought, various known methods of cultivating mycelium may be used. Any fungus that can be cultivated as mycelium may be used. Suitable fungus species for use include but are not limited to: Agaricus arvensis; Agrocybe brasiliensis; Amylomyces rouxii; Amylomyces sp.; Armillaria mellea; Aspergillus nidulans; Aspergillus niger; Aspergillus oryzae; Ceriporia lacerata; Coprinus comatus; Fibroporia vaillantii; Fistulina hepatica; Flammulina velutipes; Fomitopsis officinalis; Ganoderma sessile; Ganoderma tsugae; Hericium erinaceus; Hypholoma capnoides; Hypholoma sublaterium; Inonotus obliquus; Lactarius chrysorrheus; Macrolepiota procera; Morchella angusticeps; Myceliophthora thermophila; Neurospora crassa; Penicillium camembertii; Penicillium chrysogenum; Penicillium rubens; Phycomyces blakesleeanus; Pleurotus djamor; Pleurotus ostreatus; Polyporus squamosus; Psathyrella aquatica; Rhizopus microspores; Rhizopus oryzae; Schizophyllum commune; Streptomyces venezuelae; Stropharia rugosoannulata; Thielavia terrestris; and Ustilago maydis. In some embodiments, the fungus used includes Ganoderma sessile, Neurospora crassa, and/or Phycomyces blakesleeanus.

In some embodiments, the strain or species of fungus may be bred to produce cultivated mycelium material with specific characteristics, such as a dense network of hyphae, a highly-branched network of hyphae, hyphal fusion within the network of hyphae, and other characteristics that may alter the properties of the cultivated mycelium material. In some embodiments, the strain or species of fungus may be genetically modified to produce cultivated mycelium material with specific characteristics.

In most embodiments, the cultivated mycelium may be grown by first inoculating a solid or liquid substrate with an inoculum of the mycelium from the selected species of fungus. In some embodiments, the substrate is pasteurized or sterilized prior to inoculation to prevent contamination or competition from other organisms. For example, a standard method of cultivating mycelium includes inoculating a sterilized solid substrate (e.g. grain) with an inoculum of mycelium. Other standard methods of cultivating mycelium include inoculating a sterilized liquid medium (e.g. liquid potato dextrose) with an inoculum of mycelium or a pure cultured spawn. In some embodiments, the solid and/or liquid substrate will include lignocellulose as a carbon source for mycelium. In some embodiments, the solid and/or liquid substrate will contain simple or complex sugars as a carbon source for the mycelium.

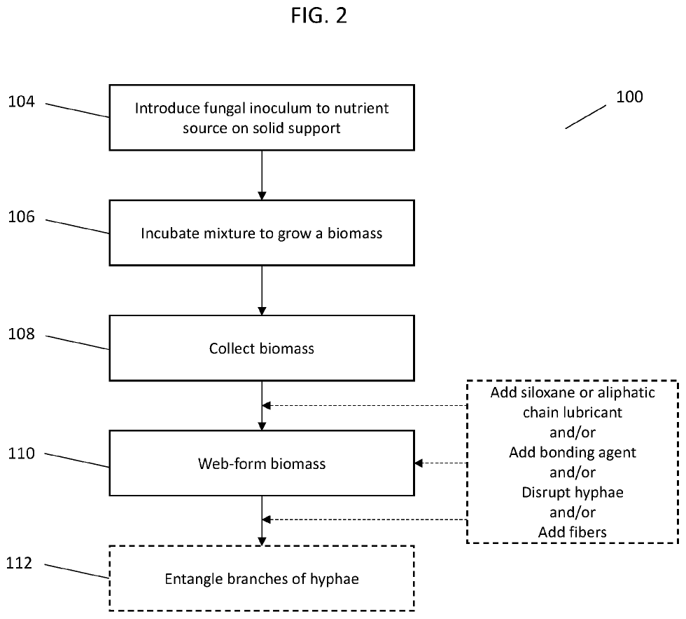

As shown in FIG. 2, a method 100 for producing a mycelium material is illustrated. The method 100 includes inoculating a nutrient source on a solid support 104, and incubating the mixture to grow a biomass of mycelium at 106, collecting the cultivated biomass of mycelium at 108, web-forming the biomass of mycelium at 110 to form a hyphal network, and optionally entangling branches of hyphae in the hyphal network at 112.

At step 106, the inoculated nutrient source is incubated to promote growth of the mycelium biomass. The conditions of the nutrient source and solid support can be selected to promote growth of a mycelium biomass having a plurality of branches of hyphae having sufficient morphological characteristics for entanglement in a downstream process. Exemplary morphological characteristics include a minimum length of hyphae branches, a desired density of the hyphae network, a desired degree of branching of the hyphae, a desired aspect ratio, and/or a desired degree of hyphal fusion of the hyphae network. According to one aspect of the present disclosure, the conditions of the solid support in the incubating step at 106 are selected to promote growth of a biomass of mycelium having a plurality of branches of hyphae having a length of at least about 0.1 mm. For example, the hyphae can have a length of from about 0.1 mm to about 5 mm, about 0.1 mm to about 4 mm, about 0.1 mm to about 3 mm, about 0.1 mm to about 2 mm, about 0.1 mm to about 1 mm, about 1 mm to about 5 mm, about 1 mm to about 4 mm, about 1 mm to about 3 mm, about 1 mm to about 2 mm, about 2 mm to about 5 mm, about 2 mm to about 4 mm, or about 2 mm to about 3 mm.

The incubation step 106 can occur under aerobic conditions in the presence of oxygen. Optionally, the solid support can be sealed into a chamber during all or a portion of the incubation step. In some examples, oxygen may be introduced into the chamber. The incubation temperature can be selected based on the specific fungal species. In some examples, the temperature of the chamber during incubation is from about 20° C. to about 40° C., about 25° C. to about 40° C., about 30° C. to about 40° C., about 35° C. to about 40° C., about 20° C. to about 35° C., about 25° C. to about 35° C., about 30° C. to about 35° C., about 20° C. to about 30° C., or about 25° C. to about 30° C.

The incubation step 106 is configured to promote the growth of a biomass of mycelium that includes a plurality of branches of hyphae. The incubation step 106 can be ended when the cultivated biomass of mycelium is collected at step 108. The incubation step 106 may be ended at a predetermined time or when a predetermined concentration of mycelium biomass is reached. There may be some continued growth of the mycelium after the cultivated biomass is collected at step 108. Optionally, the mycelium biomass may be treated to stop growth of the mycelium.

At step 108 the cultivated mycelium biomass is collected. The collected biomass can be made into a slurry by adding the dry mycelium biomass to an aqueous solution. At step 108 a concentration of the collected biomass of mycelium in such a slurry may be adjusted based on the subsequent web-forming process at step 110. In some examples, the cultivated biomass of mycelium is in the form of slurry. The concentration of the biomass of mycelium may be adjusted by increasing a volume of the slurry or concentrating the mycelium biomass by removing at least a portion of the liquid from the slurry. In some examples, the concentration of the mycelium biomass may be adjusted to a concentration of from about 10 g/L to about 30 g/L, about 10 g/L to about 25 g/L, or about 10 g/L to about 20 g/L. In other examples, the cultivated biomass of mycelium may be collected and dried.

In some aspects, a lubricant can optionally be added to the cultivated biomass of mycelium before, during, or after the web-forming process at step 110. The lubricant can be added before, during, or after collecting the cultivated biomass of mycelium and/or adjusting the concentration of the cultivated biomass of mycelium. The lubricant can be any lubricant described here, such as a siloxane or an aliphatic chain compound. For example, a siloxane lubricant can be, but is not limited to, a hydroxysilicone, a silicone hydride, an epoxy silicone, an aminosilicone, or an alkyl ethylene oxide condensate. An aliphatic chain compound lubricant can be, but is not limited to, 2-octenyl succinic anhydride (OSA), 2-dodecenyl succinic anhydride, octadecenyl succinic anhydride, 3-Chloro-2-hydroxypropyldimethyldodecylammonium chloride, heptanoic anhydride, butyric anhydride, stearic anhydride, or a chlorohydrin.

A bonding agent can also optionally be added to the cultivated biomass of mycelium before, during, or after the web-forming process at step 110. The bonding agent can be added with the lubricant, before the lubricant, or after the lubricant. The bonding agent can include any vinyl acetate-ethylene copolymer, vinyl acetate-acrylic copolymer, adhesive, resin, cross-linking agent, or polymeric matrix material described herein and combinations thereof.

In some aspects, the plurality of branches of hyphae can optionally be disrupted, before, during, or after the web-forming process at step 110. The plurality of branches of hyphae can be disrupted according to any of the mechanical and/or chemical methods described herein for disrupting hyphae. For example, prior to the web-forming process at step 110, the hyphae can mechanically disrupted by a mechanical action such as blending, chopping, impacting, compacting, bounding, shredding, grinding, compressing, high-pressure waterjet, or shearing forces. The hyphae can be disrupted before, during, or after adjusting the concentration of the cultivated biomass of mycelium.

In some aspects, the collected biomass of mycelium can optionally be pressed before or after adding the lubricant and/or bonding agent.

In some aspects, the collected biomass of mycelium can optionally be combined with natural and/or synthetic fibers, before, during, or after the web-forming process at step 110. In one aspect, the fibers can be combined with the mycelium before, during, or after disrupting the plurality of branches of hyphae. The fibers can have any suitable dimension. Non-limiting examples of suitable fibers include cellulosic fibers, cotton fibers, rayon fibers, Lyocell fibers, TENCEL™ fibers, polypropylene fibers, and combinations thereof. In one aspect, the fibers can have a length of less than about 25 mm, less than about 20 mm, less than about 15 mm, or less than about 10 mm. For example, the fibers can have a length of from about 1 mm to about 25 mm, about 1 mm to about 20 mm, about 1 mm to about 15 mm, about 1 mm to about 10 mm, about 1 mm to about 5 mm, about 5 mm to about 25 mm, about 5 mm to about 20 mm, about 5 mm to about 15 mm, about 5 mm to about 10 mm, about 10 mm to about 25 mm, about 10 mm to about 20 mm, or about 10 mm to about 15 mm. The fibers may be combined with the mycelium in a desired concentration. In one example, the fibers may be combined with the mycelium in an amount of from about 1 wt % to about 25 wt %, about 1 wt % to about 20 wt %, about 1 wt % to about 15 wt %, about 1 wt % to about 10 wt %, about 1 wt % to about 5 wt %, about 5 wt % to about 25 wt %, about 5 wt % to about 20 wt %, about 5 wt % to about 15 wt %, about 5 wt % to about 10 wt %, about 10 wt % to about 25 wt %, about 10 wt % to about 20 wt %, or about 10 wt % to about 15 wt %.

At step 110, the biomass of mycelium collected in step 108 can be treated according to a web-forming process to form a hyphal network. The web-forming process can include any of the wet-lay, dry array, or air-lay techniques described herein. The hyphae of the web formed in step 110 can optionally be chemically and/or thermally bonded using any of the bonding agents described herein.

Optionally, the web-forming at step 110 can include laying the branches of hyphae on a supporting material. As described herein, in some aspects the supporting material is a reinforcing material. Non-limiting examples of a suitable supporting material include a woven fiber, a mass of contiguous, disordered fibers (e.g., non-woven fibers), perforated material (e.g., a metal mesh or perforated plastic), a mass of discontinuous particles (e.g., pieces of woodchip), a cheesecloth, a fabric, a knot fiber, a scrim, and a textile. The hyphae can be combined with, contacted with, and/or incorporated into the supporting material. For example, in some aspects, the hyphae can be woven, twisted, would, folded, entwined, entangled, and/or braided together with the supporting material to form a mycelium material, as described herein. In some aspects, the fibers can be laid on the supporting material before, during, and/or after adding a chemical bonding agent. In some aspects, a reinforcing material can be combined with the branches of hyphae before, during, or after the web-forming step 110.

At step 112, the hyphal network formed at step 110 can undergo an entanglement process to entangle the plurality of branches of hyphae in the hyphal network. The entanglement process can include needle punching (also referred to as felting) and/or hydroentangling. When a supporting material is present, the entanglement process optionally includes entangling at least a portion of the plurality of hyphae branches with the supporting material. The entanglement process can form mechanical interactions between hyphae and optionally between hyphae and a supporting material (when present). In some embodiments, the hyphae are not entangled with a supporting material.

In some aspects, the entanglement at step 112 is achieved through a needle punching or needle felting process in which one or more needles are passed into and out of the hyphal network. Movement of the needles in and out of the hyphal network facilitate entangling the hyphae and optionally orienting the hyphae. A needle punch having an array of needles can be used to punch the hyphal network at a plurality of locations with each pass of the needle array. The number of needles, spacing of needles, shape of the needles, and size of the needles (i.e., needle gauge) can be selected to provide the desired degree of entanglement of the hyphal network. For example, the needles may be barbed and have any suitable shape, non-limiting examples of which include a pinch blade, a star blade, and a conical blade. The number of needle punches per area and the punching rate can also be selected to provide the desired degree of entanglement of the hyphal network. The parameters of the needle punching or needle felting process can be selected at least based in part on the fungal species, the morphology and dimensions of the hyphae forming the hyphal network, the desired degree of entanglement, and/or end-use applications of the mycelium material.

In some aspects, the entanglement at step 112 is achieved through a hydroentanglement process. The hydroentanglement process directs high pressure liquid jets into the hyphal network to facilitate entangling the hyphae. The liquid may be any suitable liquid, an example of which includes water. The entanglement process can include a spinneret having an array of holes configured to direct a stream of liquid at a specific location in the hyphal network. The diameter of the holes can be selected to provide a jet of liquid having the desired diameter to direct at the hyphal network. Additional aspects of the spinneret, such as the number of holes in the array and the spacing of the holes in the array can be selected to provide the desired degree of entanglement of the hyphal network. The hyphal network and the spinneret may move relative to one another such that the liquid jets are directed at the hyphal network in a pattern. For example, the spinneret may move relative to the hyphal network in a generally “Z” or “N” shaped pattern to provide multiple passes of the spinneret over the hyphal network. The number of passes and the application pattern can be selected to provide the desired degree of entanglement of the hyphal network. The parameters of the hydroentanglement process can be selected based at least in part on the fungal species, the morphology and dimensions of the hyphae forming the hyphal network, the desired degree of entanglement, and/or end-use applications of the mycelium material. In some examples, the hydroentanglement process occurs in phases in which a portion of the mycelium material is web-formed (e.g., wet-laying), the hydroentanglement process proceeds, and then a second portion of the mycelium material is web-formed on top of the first portion and the hydroentanglement process is repeated. This process of web-forming a portion of the mycelium material and hydroentangling the web-formed portion can be repeated any number of times until a final thickness of material is web-formed.

The liquid pressure, the diameter of the openings in the spinneret, and/or the flow rate of liquid can be selected to provide the desired degree of entanglement of the hyphal network and optionally entanglement of the hyphal network and a supporting material. For example, the liquid pressure during the hydroentanglement process can be at least 100 psi, at least 200 psi, at least 300 psi, at least 400 psi, at least 500 psi, at least 600 psi, at least 700 psi, at least 800 psi, at least 900 psi, or at least 1000 psi. In some examples, the liquid jet pressure is from about 700 to about 900 psi. In some examples, the diameter of the openings in the spinneret is at least about 10 microns, at least about 30 microns, at least about 50 microns, at least about 70 microns, at least about 90 microns, at least about 110 microns, at least about 130 microns, or at least about 150 microns. For example, the diameter of the openings in the spinneret can be from about 10 microns to about 150 microns, from 20 microns to about 70 microns, about 30 microns to about 80 microns, about 40 microns to about 90 microns, about 50 microns to about 100 microns, about 60 microns to about 110 microns, or about 70 microns to about 120 microns. In some examples, the openings have a diameter of about 50 microns. The flow rate of liquid can be from about 100 mL/min. to about 300 mL/min. in some examples. In some examples, the belt speed during the entanglement process is about 1 meter/minute.

After completion of the entanglement process at 112, the mycelium material can be processed according to any of the post-processing methods and/or treatments described herein. Non-limiting examples of post-processing methods and treatments include treatment with a plasticizer, treatment with a tannin and/or dye, treatment with a preservative, treatment with a protein source, treatment with a coating and/or finishing agent, a drying process, a rolling or pressing process, and treatment in an embossing process.

In various embodiments, the liquid or solid substrate may be supplemented with one or more different nutritional sources. The nutritional sources may contain lignocellulose, simple sugars (e.g. dextrose, glucose), complex sugars, agar, malt extract, a nitrogen source (e.g. ammonium nitrate, ammonium chloride, amino acids) and other minerals (e.g. magnesium sulfate, phosphate). In some embodiments, one or more of the nutritional sources may be present in lumber waste (e.g. sawdust including from hardwoods, beeches, and hickory) and/or agricultural waste (e.g. livestock feces, straw, corn stover). Once the substrate has been inoculated and, optionally, supplemented with one or more different nutritional sources, cultivated mycelium may be grown. Methods of growing mycelium have been well established in the art. Exemplary methods of growing mycelium include but are not limited to U.S. Pat. Nos. 5,854,056; 4,960,413; and 7,951,388.

In some embodiments, the growth of the cultivated mycelium will be controlled to prevent the formation of fruiting bodies. Various methods of preventing fruiting body formation as discussed in detail in U.S. Patent Publication No. 2015/0033620; U.S. Pat. Nos. 9,867,337; and 7,951,388. In other embodiments, the cultivated mycelium may be grown so that it is devoid of any morphological or structural variations. Depending on the embodiment sought, growing conditions such as exposure to light (e.g. sunlight or a growing lamp), temperature, carbon dioxide may be controlled during growth.

In some embodiments, the cultivated mycelium may be grown on an agar medium. Nutrients may be added to the agar/water base. Standard agar media commonly used to cultivate mycelium material include, but are not limited to, a fortified version of Malt Extract Agar (MEA), Potato Dextrose Agar (PDA), Oatmeal Agar (OMA), and Dog Food Agar (DFA).

In most embodiments, the cultivated mycelium material may be grown as a solid mass and may later be disrupted. Cultivated mycelium material that is disrupted may be a live mat, preserved, or otherwise treated to kill the mycelium (i.e., stop mycelium growth) as described below.

In some embodiments, cultivated mycelium material may be grown to include elongate hyphae defining fine filaments that interconnect with one another, and further may interconnect with various supporting materials provided in a growing procedure, as further described below. The fine filaments may be analyzed using an optical magnifying or imaging device to determine if a grown length of the fine filaments is adequate to support sufficient network interconnection between the fine filaments and various additives. The fine filaments should not only be of a sufficient length, but also flexible to provide adequate interconnection therebetween.

In some embodiments, cultivated mycelium material may be processed using a dry array, a wet-lay, or an air-lay technique. In dry-lay or dry array, an inert or growing mycelium network of branched hyphae may be pulled apart and detangled to expand the volume of the network. Similarly, in a wet-lay technique, an inert or growing mycelium network of branched hyphae may be saturated in a liquid medium to detangle and expand the volume of the network. Further, in an air-lay technique, an inert or growing mycelium network of branched hyphae may be suspended in air to create a web that expands the volume of the network. After such a technique, the expanded network can be compressed to provide a dense or compacted network. The web can be densified to include an overall density profile of at least 6 gm per cubic meter. A compacted web can be embossed with a replicated leather pattern for providing a leather alternative material.

In some embodiments, the method comprises a step of web-forming the collected biomass of the mycelium. In some embodiments, the step of web-forming the collected biomass of mycelium comprises depositing the biomass of mycelium on a supporting material.

In some embodiments, the supporting material comprises a woven fiber, a non-woven fiber, a mesh, a perforated plastic, woodchips, a cheesecloth, a fabric, a knot fiber, a scrim, a textile, or combinations thereof.

In some embodiments, the entangling the plurality of branches of hyphae comprises entangling at least a portion of the plurality of branches of hyphae with the supporting material.

In some embodiments, the method further comprises combining a reinforcing material with the biomass of mycelium one of prior to the web-forming step, during the web-forming step, or after the web-forming step. In some embodiments, web-forming comprises wet-laying, air-laying, or dry-laying.

In some embodiments, the method further comprises combining one of natural fibers, synthetic fibers, or a combination thereof with the biomass of mycelium one of prior to the web-forming step, during the web-forming step, or after the web-forming step.

In some embodiments, the fibers have a length of less than 25 millimeters.

Disrupted Cultivated Mycelium Material

Various types of cultivated mycelium material including one or more masses of branching hyphae may be disrupted at a variety of points during the production process, thus generating one or more masses of disrupted branching hyphae. In such embodiments, the cultivated mycelium material comprises one or more masses of disrupted branching hyphae. The cultivated mycelium material may be disrupted before or after adding a bonding agent. In one aspect, the cultivated mycelium material may be disrupted at the same time as adding a bonding agent. Exemplary embodiments of disruptions include, but are not limited to, mechanical action, chemical treatment, or a combination thereof. For example, the one or more masses of branching hyphae may be disrupted by both a mechanical action and chemical treatment, a mechanical action alone, or chemical treatment alone.

In some embodiments, the one or more masses of branching hyphae is disrupted by a mechanical action. Mechanical actions may include blending, chopping, impacting, compacting, bounding, shredding, grinding, compressing, high-pressure, waterjet, and shearing forces. In some embodiments, the mechanical action includes blending the one or more masses of branching hyphae. Exemplary methods of achieving such a disruption include use of a blender, a mill, a hammer mill, a drum carder, heat, pressure, liquid such as water, a grinder, a beater, and a refiner. In an exemplary production process, a cultivated mycelium material is mechanically disrupted by a conventional unit operation, such as homogenization, grinding, coacervation, milling, jet milling, waterjet and the like.

According to a further aspect, the mechanical action includes applying a physical force to the one or more masses of branching hyphae such that at least some of the masses of branching hyphae are aligned in a particular formation, e.g., aligned in a parallel formation, or along or against the stress direction. The physical force can be applied to one or more layers of a cultivated mycelium material or composite mycelium material. Such disrupted mycelia material can typically be constructed with layers with varying orientation. Exemplary physical forces include, but are not limited to, pulling and aligning forces. Exemplary methods of achieving such a disruption include use of rollers and drafting equipment. In some embodiments, a physical force is applied in one or more directions such that the at least some of the masses of branching hyphae are aligned in parallel in one or more directions, wherein the physical force is applied repeatedly. In such embodiments, the physical force may be applied at least two times, e.g., at least three times, at least four times, or at least five times.

In some other embodiments, the one or more masses of branching hyphae is disrupted by chemical treatment. In such embodiments, the chemical treatment includes contacting the one or more masses of branching hyphae with a base or other chemical agent sufficient to cause a disruption including, but not limited to alkaline peroxide, beta-glucanase, surfactants, acids, and bases such as sodium hydroxide and sodium carbonate (or soda ash). The pH of the cultivated mycelium material in solution can be monitored for the purpose of maintaining the optimal pH.

In some embodiments, the disruptions described herein generate one or more masses of disrupted branching hyphae, e.g., sub-networks. As used herein, a “sub-network” refers to discrete masses of branching hyphae that are produced after disruption, e.g., a mechanical action or chemical treatment. A sub-network may come in a wide assortment of shapes, e.g., sphere-, square-, rectangular-, diamond-, and odd-shaped sub-networks, etc., and each sub-network may come in varied sizes. The cultivated mycelium material may be disrupted sufficiently to produce one or more masses of disrupted branching hyphae, e.g., sub-networks, having a size in the desired ranges. In many instances, the disruption can be controlled sufficiently to obtain both the size and size distribution of the sub-network within a desired range. In other embodiments, where more precise size distributions of sub-networks are required, the disrupted cultivated mycelium material can be further treated or selected to provide the desired size distribution, e.g. by sieving, aggregation, or the like. For example, a sub-network may have a size represented by, e.g., length, of about 0.1 mm to about 5 mm, inclusive, e.g., of about 0.1 mm to about 2 mm, about 1 mm to about 3 mm, about 2 mm to about 4 mm, and about 3 mm to about 5 mm. In some embodiments, a sub-network may have a size represented by a length of about 2 mm. The “length” of a sub-network is a measure of distance equivalent to the most extended dimension of the sub-network. Other measurable dimensions include, but are not limited to, length, width, height, area, and volume.

In various embodiments, physical force may be used to create new physical interactions (i.e. re-entangle) between the one or more masses of branching hyphae after disruption. Various known methods of creating entanglements between fiber may be used, including methods of creating non-woven materials by creating mechanical interactions between fibers. In some embodiments described below, hydroentanglement may be used to create mechanical interactions between the hyphae after the hyphae have disrupted.

Preserved Cultivated Mycelium Material

Once the cultivated mycelium material has been grown, it may be optionally separated from the substrate in any manner known in the art, and optionally subjected to post-processing in order to prevent further growth by killing the mycelium and otherwise rendering the mycelium imputrescible, referred to herein as “preserved mycelium material”. Suitable methods of generating preserved mycelium material can include drying or desiccating the cultivated mycelium material (e.g. pressing the cultivated mycelium material to expel moisture) and/or heat treating the cultivated mycelium material.

In a specific embodiment, the cultivated mycelium material is pressed at 190,000 pounds force to 0.25 inches for 30 minutes. The cultivated mycelium material can be pressed by at least 100, 1000, 10,000, 100,000, 110,000, 120,000, 130,000, 140,000, 150,000, 160,000, 170,000, 180,000, 190,000, 200,000, or 300,000 or more pounds force. The cultivated mycelium material can be pressed to at least 0.1, 0.11, 0.12, 0.13, 0.14, 0.15, 0.16, 0.18, 0.19, 0.2, 0.21, 0.22, 0.23, 0.24, 0.25, 0.26, 0.27, 0.28, 0.29, 0.3, 0.31, 0.32, 0.33, 0.35, 0.36, 0.37, 0.38, 0.39, 0.4, 0.41, 0.42, 0.43, 0.44, 0.45, 0.46, 0.47, 0.48, 0.49, 0.5, 0.52, 0.53, 0.54, 0.55, 0.56, 0.57, 0.58, 0.59, 0.6, 0.61, 0.62, 0.63, 0.64, 0.65, 0.66, 0.67, 0.69, 0.7, 0.71, 0.72, 0.73, 0.74, 0.75, 0.76, 0.77, 0.78, 0.79, 0.8, 0.81, 0.82, 0.83, 0.84, 0.86, 0.87, 0.88, 0.89, 0.9, 0.91, 0.92, 0.93, 0.94, 0.95, 0.96, 0.97, 0.98, 0.99, or 1 inch or more. The cultivated mycelium material can be pressed to at least 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 centimeter or more. The cultivated mycelium material can be pressed for at least 1 min, 5 min, 10 min, 15 min, 20 min, 25 min, 30 min, 35 min, 40 min, 45 min, 50 min, 55 min, or 60 min or more.

Suitable methods of drying organic matter to render it imputrescible are well known in the art. In some embodiments, the cultivated mycelium material is dried in an oven at a temperature of 100° F. or higher. In other embodiments, the cultivated mycelium material is heat pressed.

In other instances, living or dried cultivated mycelium material is processed using one or more solutions that function to remove waste material and water from the mycelium. In some embodiments, the solutions include a solvent such as ethanol, methanol or isopropyl alcohol. In some embodiments, the solutions include a salt such as calcium chloride. Depending on the embodiments, the cultivated mycelium material may be submerged in the solution for various durations of time with and without pressure. In some embodiments the cultivated mycelium material may be submerged in several solutions consecutively. In a specific embodiment, the cultivated mycelium material may first be submerged in one or more first solutions including an alcohol and a salt, then submerged in a second solution including alcohol. In another specific embodiment, the cultivated mycelium material may first be submerged in one or more first solutions including an alcohol and a salt, then submerged in a second solution including water. After treatment with solution, the cultivated mycelium material may be pressed using a hot or cold process and/or dried using various methods including air drying and/or vacuum drying. U.S. Patent Publication No. 2018/0282529, the entirety of which is incorporated herein by reference, describes these embodiments in detail.

In one aspect, the cultivated mycelium material may be fixated by adjusting pH using an acid such as formic acid. In specific embodiments, the pH will be at least 2, 3, 4 or 5. In some embodiments, the pH of the cultivated mycelium material will be adjusted to an acidic pH of 3 in order to fix the cultivated mycelium material using various agents such as formic acid. In specific embodiments, the pH will be adjusted to a pH less than 6, 5, 4 or 3 in order to fix the cultivated mycelium material. In one embodiment, the pH will be adjusted to a pH of 5.5.

### Lubricants

Various lubricants may be applied to the cultivated mycelium material or composite mycelium material during production to alter the mechanical properties of the cultivated mycelium material or composite mycelium material. The role of a lubricant is to, without intending to be bound by theory, decrease the crystallinity from tightly packed hydrogen-bonding networks formed within the substructures of the hyphae, thereby increasing the internal lubrication of the hyphae and the flexibility of the cultivated mycelium material. Additionally, lubricants with various charges such as quaternary ammonium or carboxylate moieties in the added group may beneficially impact interactions with binding agents, fat liquors, and/or reactive dyes, each of which can themselves be charged. For instance, lubricants may increase the hydrophobic interactions between the lubricant side-chains and any later added fat liquors. Aliphatic chain compound lubricants can also be used to adjust the cultivated mycelium material's hydrophilicity or hydrophobicity. The type and amount of lubricant used in the present disclosure depend on what properties are desired. In various embodiments, an effective amount of lubricant may be used. As used herein, an “effective amount” with respect to a lubricant refers to the amount of lubricant that is sufficient to provide added flexibility and/or other properties such as additional softness, strength, durability, and compatibility.

Without intending to be bound by theory, due to their medium or large polymeric size, siloxanes may become trapped within the hyphae network when added to disrupted mycelium material or the pressed or unpressed mycelium material. The trapped siloxane molecules may unable to leach out of the hyphae network of the material and thereby increase the flexibility of the material due to internal lubrication of the hyphae networks, e.g., by decreasing the crystallinity and brittleness of the of the cultivated mycelium material. Siloxanes may also bind to the hyphae chitin and thereby decrease crystallinity and brittleness of the cultivated mycelium material. Treatment with siloxanes may also leave cyclic siloxanes and free isocyanate residues in the hyphae network, providing additional reactive groups that can later be reacted with additional treatment compounds.

In general lubricants are added during the initial production of the cultivated mycelium material, e.g., during mechanical disruption, web forming, wet lay, pressing, or before drying a newly made mycelium panel.

Lubricants such as siloxanes and aliphatic chain compounds provide an improvement over the traditional fat liquors that have been used as finishing products to provide softness and flexibility. First, the lubricants can be added early in the process of making the material. For instance, the lubricants can be added to the disrupted mycelium material before or during a wet lay process, or before or during a pressing process. This results in better uptake, retention, and permeation of the lubricant into the material, as compared to soaking a processed and dried cultivated material with a fat liquor in a post-processing step. Second, adding the lubricant at the early step of wet laying, web forming, or pressing traps the lubricant within the mycelium material and reduces later leaching of the lubricant from the material. The aliphatic chain compounds thus are covalently linked to the hyphae in a more uniform manner, while the siloxanes are more thoroughly and uniformly trapped within the hyphae network. This results in less leaching of the lubricant out of the material, as compared to fat liquor leaching from less well permeated mycelium material. Third, the addition of the lubricant at the early stage prior to or with the mycelium disruption or pressing step removes the later fat liquor processing step and thus makes the production process faster. Fourth, post-processing with fat liquor requires a significant amount of water to dilute the fat liquor in order to soak the mycelium material. Addition of the lubricant to a slurry of disrupted mycelium material reduces the amount of water required in the production process.

Siloxanes are compounds with functional groups with an Si—O—Si linkage. Siloxanes can also comprise branched compounds with pairs of silicon centers separated with one oxygen atom. Siloxane functional groups form silicones, such as polydimethylsiloxane. Siloxanes are hydrophobic, have low thermal conductivity and high flexibility. Exemplary siloxane compounds that can be used in a cultivated mycelium material include siloxane products from Starchem and Wacker. Any Starchem or Wacker siloxane can be used in the mycelium material disclosed herein, including, but not limited to, StarSoft GA, StarPel 366, StarChem 2543, StarSoft HS 20, StarSoft HS 40, Reactosil RWS, StarSoft WAM, StarSoft Bis 45, StarSoft TS-T3, or Wacker Elastosil products. Starchem siloxanes can also comprise mixtures of siloxanes and polyurethanes.

Aliphatic chains are open chain hydrocarbons, where the hydrocarbon chain contains no aromatic rings. Aliphatic compounds are also known as non-aromatic hydrocarbons. An aliphatic chain compound can have at least 2 carbons, at least 3 carbons, at least 4 carbons, at least 5 carbons, at least 6 carbons, at least 7 carbons, at least 8 carbons, at least 9 carbons, at least 10 carbons, at least 11 carbons, at least 12 carbons, at least 13 carbons, at least 14 carbons, at least 15 carbons, at least 16 carbons, at least 17 carbons, at least 18 carbons, at least 19 carbons, at least 20 carbons, at least 21 carbons, at least 22 carbons, at least 23 carbons, at least 24 carbons, at least 25 carbons, at least 26 carbons, at least 27 carbons, at least 28 carbons, at least 29 carbons, or at least 30 or more carbons. As contemplated herein, a useful aliphatic chain compound for use as a lubricant comprises an aliphatic hydrocarbon chain. As contemplated herein, a useful long aliphatic chain compound for use as a lubricant comprises an aliphatic hydrocarbon chain with at least 8 carbons. An aliphatic chain compound lubricant can be, but is not limited to, 2-octenyl succinic anhydride (OSA), 2-dodecenyl succinic anhydride, octadecenyl succinic anhydride, stearic anhydride, 3-Chloro-2-hydroxypropyldimethyldodecylammonium chloride, heptanoic anhydride, butyric anhydride, a chlorohydrin, C-12 succinic anhydride, C-18 succinic anhydride, or fatty acid anhydride of various chain lengths ranging from C-7 heptanoic anhydride to C-18 stearic anhydride succinic anhydride. For example, alkenyl succinic anhydrides with side-chain lengths of 8-18 carbons can be used. Any aliphatic chain with sufficient carbon chain length to render the resulting preferred mechanical properties can be used.

In some embodiments, the aliphatic chain compound is hydrophobic. Hydrophobicity of an aliphatic chain compound increases as the number of carbons in the hydrocarbon chain increases. Thus, a C-18 hydrocarbon is more hydrophobic than a C-7 hydrocarbon.

The present disclosure is not limited to the above lists of suitable lubricants. Other lubricants are known in the art.

The lubricant can be added to cultivated mycelium material that has been pressed, had one or more masses of hyphae disrupted, and/or hydroentangled. The lubricant can be added to cultivated mycelium material before disruption of the one or more masses of branching hyphae or pressing. The lubricant can be added to cultivated mycelium material during disruption of the one or more masses of branching hyphae or pressing. The lubricant can be added to cultivated mycelium material after disruption of the one or more masses of branching hyphae or pressing. In some embodiments, a pressed cultivated mycelium material comprises a lubricant, wherein the pressed cultivated mycelium material does not comprise a fat liquor. In some embodiments, a disrupted cultivated mycelium material comprises a lubricant, wherein the pressed cultivated mycelium material does not comprise a fat liquor.

In some embodiments, a pressed cultivated mycelium material is contacted with a lubricant. In some embodiments, a disrupted cultivated mycelium material is contacted with a lubricant. In some embodiments, the lubricant is added before the masses of branching hyphae are disrupted. In some embodiments, the lubricant is added during the disruption of the one or more masses of branching hyphae. In some embodiments, the lubricant is added after the masses of branching hyphae are disrupted. In some embodiments, the lubricant is added before the cultivated mycelium material is pressed. In some embodiments, the lubricant is added during the pressing of the cultivated mycelium material. In some embodiments, the lubricant is added after the cultivated mycelium material is pressed.

In some embodiments, the lubricant is an aliphatic chain compound that binds covalently to the hyphae. In some embodiments, the aliphatic chain compound binds covalently to at least one hydroxyl group on a hyphae of a cultivated mycelium material. In some embodiments, the aliphatic chain compound binds covalently to at least one carboxyl group on a hyphae of a cultivated mycelium material. In some embodiments, the aliphatic chain compound binds covalently to at least one amino group on a hyphae of a cultivated mycelium material. In some embodiments, an aliphatic chain compound modifies interaction with binding agents, fatliquors, and/or dyes.

### Bonding Agents

Various aspects of the present disclosure include a bonding agent. A “bonding agent” as used herein may include any suitable agent that provides added strength and/or other properties such as additional softness, strength, durability, and compatibility. A bonding agent may be an agent that reacts with some portion of the cultivated mycelium material, enhances the treatment of the cultivated mycelium material, co-treated with the cultivated mycelium material or treated separately, but as a network with the cultivated mycelium material, to produce a composite mycelium material. In some aspects, a bonding agent is added prior to the disruption. In other aspects, a bonding agent is added after the disruption. In some other aspects, a bonding agent is added while the sample is being disrupted. Bonding agents include an adhesive, a resin, a crosslinking agent, and/or a matrix. A composite mycelium material described herein includes cultivated mycelium material and bonding agents that may be water-based, 100% solids, UV and moisture cure, two-component reactive blend, pressure sensitive, self-crosslinking hot melt, and the like.

In some embodiments, the bonding agent is selected from the group including a natural adhesive or a synthetic adhesive. In such embodiments, the natural adhesive may include a natural latex-based adhesive. In specific embodiments, the natural latex-based adhesive is leather glue or weld. The bonding agents may include anionic, cationic, and/or non-ionic agents. In one aspect, the bonding agents may include crosslinking agents.

In some embodiments, the bonding agent has a particle size of less than or equal to 1 μm, a sub-zero glass transition temperature, or a self-crosslinking function. In some embodiments, the bonding agent has a particle size of less than or equal to 1 μm, a sub-zero glass transition temperature, and a self-crosslinking function. In some embodiments, the bonding agent has a particle size of less than or equal to 1 μm. In some embodiments, the bonding agent has a sub-zero glass transition temperature. In some embodiments, the bonding agent has a self-crosslinking function. In some embodiments, the bonding agent has a particle size of less than or equal to 500 nanometers. Specific exemplary bonding agents include vinyl acetate ethylene copolymers such as Dur-O-Set® Elite Plus and Dur-O-Set® Elite 22.

In some embodiments, the bonding agent has a glass transition temperature of −100-−10° C.-100-−90° C., −90-−80° C., −80-−70° C., −70-−60° C., −60-−50° C., −50-−40° C., −40-−30° C., −30-−20° C., −20-−10° C., −10-−10° C., −30-−25° C., −25-−20° C., −20-−15° C., −15-−10° C., −10-−5° C., −5-−0° C., −90° C., −80° C., −70° C., −60° C., −50° C., −40° C., −35° C., −30° C., −25° C., −20° C., −15° C., −10° C., −5° C., or 0° C. In some embodiments, the bonding agent has a glass transition temperature of −15° C.

Other exemplary bonding agents include, but are not limited to transglutaminase, polyamide-epichlorohydrin resin (PAE), citric acid, genipin, alginate, vinyl acetate-ethylene copolymers, and vinyl acetate-acrylic copolymers. In some embodiments, the binder is polyamide-epichlorohydrin resin (PAE). In some embodiments, the binder is a vinyl acetate-ethylene copolymer. In some embodiments, the binder is a vinyl acetate-acrylic copolymer.

In some embodiments, the bonding agent includes one or more reactive groups. For example, the bonding agent reacts with active hydrogen containing groups such as amine, hydroxyl, and carboxyl groups. In a specific embodiment, the bonding agent crosslinks one or more masses of branching hyphae via the one or more reactive groups. In some instances, amines are present on chitin, and hydroxyl and carboxyl groups are present on the polysaccharides and proteins surrounding the chitin. In a specific embodiment, PAE includes cationic azetidinium groups. In such embodiments, the cationic azetidinium groups on PAE act as reactive sites in the polyamideamine backbone, and react with active hydrogen containing groups such as amine, hydroxyl, and carboxyl groups, in the one or more branches of hyphae.

Further examples of bonding agents include, but are not limited to, citric acid in combination with sodium hypophosphite or monosodium phosphate or sodium dichloroacetate, alginate in combination with sodium hypophosphite or monosodium phosphate or sodium dichloroacetate, epoxidized soybean oil, N-(3-Dimethylaminopropyl)-N′-ethylcarbodiimide hydrochloride (EDC), polyamide epichlorohydrin resin (PAE), and ammonium persulfate. Some examples of bonding agents include epoxies, isocyanates, sulfur compounds, aldehydes, anhydrides, silanes, aziridines, and azetidinium compounds and compounds with all such functional groups. Possible formaldehyde-containing bonding agents include formaldehyde, phenol formaldehyde, urea formaldehyde, melamine urea formaldehyde, melamine formaldehyde, phenol resorcinol and any combinations of them.

Additional examples of suitable bonding agents include latex materials, such as butadiene copolymers, acrylates, vinyl-acrylics, styrene-acrylics, styrene-butadiene, nitrile-butadiene, polyvinyl acetates, olefin containing polymers, e.g., vinyl acetate-ethylene copolymers, vinyl ester copolymers, halogenated copolymers, e.g., vinylidene chloride polymers. Latex-based agents, when used, can contain functionality. Any kind of latex can be used, including acrylics. Representative acrylics include those formed from ethyl acrylate, butyl acrylate methyl (meth)acrylate, carboxylated versions thereof, glycosylated versions thereof, self-crosslinking versions thereof (for example, those including N-methyl acrylamide), and copolymers and blends thereof, including copolymers with other monomers such as acrylonitrile. Natural polymers such as starch, natural rubber latex, dextrin, lignin, cellulosic polymers, saccharide gums, and the like can also be used. In addition, other synthetic polymers, such as epoxies, urethanes, phenolics, neoprene, butyl rubber, polyolefins, polyamides, polypropylene, polyesters, polyvinyl alcohol, and polyester amides can also be used. The term “polypropylene” as used herein includes polymers of propylene or polymerizing propylene with other aliphatic polyolefins, such as ethylene, 1-butene, 1-pentene, 3-methyl-1-butene, 4-methyl-1-pentene, 4-methyl-1-hexene, 5-methyl-1-hexene and mixtures thereof. In specific embodiments, bonding agents include, but are not limited to, natural adhesives (e.g. natural latex-based adhesives such as leather glue or weld, latex, soy protein-based adhesives), synthetic adhesives (polyurethane), neoprene (PCP), acrylic copolymer, styrene-butadiene copolymer, ethylene-vinyl acetate-b, nitrocellulose, polyvinyl acetate (PVA), and vinyl acetate ethylene (VAE). In other embodiments, the bonding agent is VAE.

In one aspect, one or more bonding agents may be incorporated within the cultivated mycelium material to be bonded, either in its disrupted or undisrupted state, e.g., embedded throughout the material, or added as a thin coating layer, such as by spraying, dipping, rolling, coating, and the like, to produce a composite mycelium material. In one other aspect, one or more bonding agents may be incorporated at the same time the disruption occurs. Any suitable method of bonding may be used according to the present disclosure. Bonding of the surfaces may occur on drying, and a strong cured bond can be developed. The bonding of one or more bonding agents may include the use of open or closed-cell foam materials like urethane, olefinic rubber, and vinyl foam materials, as well as textiles, metal and fabrics in various lamination arrangements.

A bonded assembly (i.e., a laminate) may be prepared by uniformly applying the aqueous adhesive to the cultivated mycelium material. In some embodiments, the lamina includes two successive layers. In some embodiments, the lamina includes three successive layers. Various coating methods may be used such as spraying, roll coating, saturation, and the like. The coated substrate can be dried before bonding.

A composite mycelium material may be chemically bonded by impregnating the composite mycelium material with a chemical binder to link fibers to one another, including linking cellulosic fibers to one another. Non-limiting examples of suitable binders include gum arabic, vinyl acetate-ethylene (VAE), and adhesives. Examples of suitable adhesive include S-10, available from US Adhesives, U.S.A., and Bish's Original Tear Mender Instant Fabric & Leather Adhesive, available from Tear Mender, U.S.A. One example of a suitable VAE-based binder is Dur-O-Set® Elite 22, which is available from Celanese Emulsions, U.S.A. One other example of a suitable VAE-based binder is Dur-O-Set® Elite Plus, which is available from Celanese Emulsions, U.S.A. Another exemplary binder includes X-LINK® 2833, available from Celanese Emulsions, U.S.A., and which is described as a self-crosslinking vinyl acetate acrylic. In a web of interconnected hyphae, a chemical binder will have to saturate the web to diffuse through the web and reach the core of the network. Thus, a composite mycelium material may be immersed in a binder solution to fully impregnate the material. A spray application of a chemical binder may also be provided to a composite mycelium material. A spray application of a chemical binder may be aided by capillary action for dispersal, or may be aided by a vacuum application to draw the chemical binder through the material. A coater may also be used for coating a composite mycelium material.

A composite mycelium material may be bonded using a thermal bonding technique, wherein an additive is provided along with the composite mycelium material. This additive may be a “meltable” material that melts at a known heat level. The cellulosic material of the composite mycelium material does not melt, such that the composite mycelium material along with the additive can be heated to the additive's melting point. As melted, the additive can disperse within the composite mycelium material and then be cooled to harden the overall material.

The present disclosure is not limited to the above lists of suitable bonding agents. Other bonding agents are known in the art. The role of a bonding agent, regardless of type, is to, in part, provide several reactive sites per molecule. The type and amount of bonding agent used in the present disclosure depend on what properties are desired. In various embodiments, an effective amount of bonding agent may be used. As used herein, an “effective amount” with respect to a bonding agent refers to the amount of agent that is sufficient to provide added strength and/or other properties such as additional softness, strength, durability, and compatibility.

The bonding agent can be added to cultivated mycelium material that has been pressed, had one or more masses of hyphae disrupted, and/or hydroentangled. The bonding agent can be added to cultivated mycelium material before disruption of the one or more masses of branching hyphae or pressing. The bonding agent can be added to cultivated mycelium material during disruption of the one or more masses of branching hyphae or pressing. The bonding agent can be added to cultivated mycelium material after disruption of the one or more masses of branching hyphae or pressing.

In some embodiments, a pressed cultivated mycelium material is contacted with a bonding agent. In some embodiments, a disrupted cultivated mycelium material is contacted with a bonding agent. In some embodiments, the bonding agent is added before the masses of branching hyphae are disrupted. In some embodiments, the bonding agent is added during the disruption of the one or more masses of branching hyphae. In some embodiments, the bonding agent is added after the masses of branching hyphae are disrupted. In some embodiments, the bonding agent is added before the cultivated mycelium material is pressed. In some embodiments, the bonding agent is added during the pressing of the cultivated mycelium material. In some embodiments, the bonding agent is added after the cultivated mycelium material is pressed.

### Supporting Materials

According to one aspect, the cultivated mycelium material or composite mycelium material may further include a supporting material, e.g., to form a bonded assembly, i.e., a laminate. As used herein, the term “supporting material” refers to any material, or combination of one or more materials, that provide support to the cultivated mycelium material or composite mycelium material. In some embodiments, the support material is a scaffold. In some embodiments, the support material is a scrim.

In some embodiments, the supporting material is entangled within the cultivated mycelium material or composite mycelium material, e.g., a reinforcing material. In some other embodiments, the supporting material is positioned on a surface of the cultivated mycelium material or composite mycelium material, e.g., a base material. In some embodiments, the supporting material includes, but is not limited to, a mesh, a cheesecloth, a fabric, a plurality of fibers, a knit textile, a woven textile, a non-woven textile, a knit fiber, a woven fiber, a non-woven fiber, a film, a surface spray coating, and a fiber additive. In some embodiments, a knit textile is a knit fiber. In some embodiments, a woven textile is a woven fiber. In some embodiments, a non-woven textile is a non-woven fiber. In some embodiments, the supporting material may be constructed in whole or in part of any combination of synthetic fiber, natural fiber (e.g. lignocellulosic fiber), abaca fiber, metal, or plastic. The supporting material may be entangled, in part, within the cultivated mycelium material or composite mycelium material, e.g., using known methods of entanglement like felting or needle punching. In some aspects, the supporting material is not entangled within the cultivated mycelium material or composite mycelium material. Various methods known in the art may be used to form a laminate as described herein. In some other embodiments, the supporting material includes a base material that is, e.g., applied to a top or bottom surface of a cultivated mycelium material or composite mycelium material. The supporting material may be attached through any means known in the art, including, but not limited to, chemical attachment, e.g., a suitable spray coating material, in particular, a suitable adhesive, or alternatively, e.g., due to their inherent tackiness.

In some embodiments, the mycelium comprises abaca fiber or fiber additive of at least 5 wt %. In some embodiments, the mycelium comprises abaca fiber or fiber additive of at least 1 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 25 wt %, at least 30 wt %, or more.

A laminate according to the present disclosure may include at least one supporting material. If more than one supporting material is used, the cultivated mycelium material or composite mycelium material can include an inner layer of a sandwich of multiple layers, with the inner layer, e.g., being a supporting material such as a knit or woven or scaffold. In this instance, the supporting material would be embedded within the cultivated mycelium material or composite mycelium material.

Supporting materials as used herein can include scaffolds or textiles. A “scaffold” as used herein refers to any material known in the art that is distinct from the cultivated mycelium material and provides support to the cultivated mycelium material or composite mycelium material. A “scaffold” may be embedded within the cultivated mycelium material or composite mycelium material or layered on, under, or within the cultivated mycelium material or composite mycelium material. In the present disclosure, all kinds and types of scaffolds may be used, including, but not limited to films, textiles, scrims, and polymers. A “textile” as used herein refers to a type of scaffold that may be any woven, knitted, or non-woven fibrous structure. Where multiple layers are included in the cultivated mycelium material or composite mycelium material, the two or more layers may include a scaffold; or in other embodiments, the two or more layers may include a cheesecloth. Useful scaffolds include woven and non-woven scaffolds, directional and non-directional scaffolds, and orthogonal and non-orthogonal scaffolds. Useful scaffolds may include conventional scaffolds, which include a plurality of yarns oriented in the machine direction, or along the length of the scaffold, and a plurality of yarns oriented in the cross-machine direction, or across the width of the scaffold. These yarns may be referred to as the warp yarns and weft yarns, respectively. Numerous yarns can be employed including, but not limited to, fibrous materials and polymers. For example, the yarns can include, but are not limited to, fiberglass, aluminum, or aromatic polyamide polymers. In one embodiment, the scaffold includes fiberglass yarns. The scaffolds may be adhered together or locked into position using conventional bonding agents such as cross-linkable acrylic resins, polyvinyl alcohol, or similar adhesives. The scaffolds may also be mechanically entangled by employing techniques such as, but not limited to, needle punching. In yet another embodiment, the scaffolds can be locked into place by weaving. A combination of supporting materials may be used according to the present disclosure.

In some embodiments, supporting materials may be incorporated into a cultivated mycelium material or composite mycelium material as described herein according to methods known in the art, including but not limited to the methods described in U.S. Pat. Nos. 4,939,016 and 6,942,711, the entirety of which are incorporated herein by reference. For example, supporting materials may be incorporated into a cultivated mycelium material or composite mycelium material via hydroentanglement. In such embodiments, supporting materials may be incorporated into a cultivated mycelium material or composite mycelium material before or after adding a bonding agent and/or a crosslinking agent. In some embodiments, a liquid such as water directed to the cultivated mycelium material or composite mycelium material through one or more pores for hydroentanglement can pass through the cultivated mycelium material or composite mycelium material. In some embodiments, the liquid is a high-pressure liquid. In some embodiments, the pressure and water flow may vary depending, in part, on the type of supporting material and pore size. In various embodiments, the water pressure is at least 100 psi, e.g., at least 200 psi, at least 300 psi, at least 400 psi, at least 500 psi, at least 600 psi, at least 700 psi, at least 800 psi, at least 900 psi, and at least 1000 psi. In various embodiments, the water pressure is about 100 psi to about 5000 psi, inclusive, e.g., about 200 psi to about 1000 psi, about 300 psi to about 2000 psi, about 400 psi to about 3000 psi, about 500 psi to about 4000 psi, and about 600 psi to about 5000 psi. In some embodiments, the water pressure is about 750 psi. In various embodiments, the one or more pores has a diameter of at least 10 microns, e.g., at least 30 microns, at least 50 microns, at least 70 microns, at least 90 microns, at least 110 microns, at least 130 microns, and at least 150 microns. In various embodiments, the one or more pores has a diameter of about 10 microns to about 150 microns, inclusive, e.g., about 20 microns to about 70 microns, about 30 microns to about 80 microns, about 40 microns to about 90 microns, about 50 microns to about 100 microns, about 60 microns to about 110 microns, and about 70 microns to about 120 microns. In some embodiments, the one or more pores has a diameter of about 50 microns.

The cultivated mycelium material or composite mycelium material may also include auxiliary agents that are used in foam materials. Auxiliary agents or additives include crosslinking agents, processing aids (e.g., drainage aid), dispersing agent, flocculent, viscosity reducers, flame retardants, dispersing agents, plasticizers, antioxidants, compatibility agents, fillers, pigments, UV protectors, fibers such as abaca fibers, and the like. It is further contemplated that a foaming agent can be used to introduce a chemical bonding agent to a composite mycelium material. Such a foaming agent can make a web of composite mycelium material more porous by introducing air to the web.

### Plasticizers

Various plasticizers may be applied to the cultivated mycelium material or composite mycelium material to alter the mechanical properties of the cultivated mycelium material or composite mycelium material. In such embodiments, the cultivated mycelium material or composite mycelium material further includes a plasticizer. U.S. Pat. No. 9,555,395 discusses adding a variety of humectants and plasticization agents. Specifically, the U.S. Pat. No. 9,555,395 discusses using glycerol, sorbitol, triglyceride plasticizers, oils such as linseed oils, castor oils, drying oils, ionic and/or nonionic glycols, and polyethylene oxides. U.S. Patent Publication No. 2018/0282529 further discusses treating cultivated mycelium material or composite mycelium material with plasticizers such as glycerol, sorbitol or another humectant to retain moisture and otherwise enhance the mechanical properties of the cultivated mycelium material or composite mycelium material such as the elasticity and flexibility of the cultivated mycelium material or composite mycelium material. In such embodiments, the cultivated mycelium material or composite mycelium material is flexible.

In general, plasticizers are added at a later stage in producing the mycelium material, e.g., after the panel or mat has been dried and is being processed for dyeing and finishing.

Other similar plasticizers and humectants are well-known in the art, such as polyethylene glycol and fatliquors obtained by emulsifying natural oil with a liquid that is immiscible with oil (e.g. water) such that the micro-droplets of oil may penetrate the material. Various fatliquors contain emulsified oil in water with the addition of other compounds such as ionic and non-ionic emulsifying agents, surfactants, soap, and sulfate. Fatliquors may include various types of oil such as mineral, animal and plant-based oils. Appropriate fatliquors include, but are not limited to, Truposol® LEX fatliquour (Trampler, Germany), Trupon® DXV fatliquor (Trampler, Germany), Diethyloxyester dimethyl ammonium chloride (DEEDMAC), Downy fabric softener, sorbitol, m-erythritol, Tween 20 and Tween 80.

### Tannins and Dyes

In various embodiments of the present disclosure, it may be ideal to impart color to the cultivated mycelium material or composite mycelium material. As discussed in U.S. Patent Publication No. 2018/0282529, tannins may be used to impart a color to cultivated mycelium material, composite mycelium material, or preserved composite mycelium material.

As cultivated mycelium material and/or composite mycelium material includes, in part, of chitin, it lacks the functional sites that are abundant in protein-based materials. Therefore, it may be necessary to functionalize the chitin in the cultivated mycelium material or composite mycelium material in order to create binding sites for acid and direct dyes. Methods of functionalizing chitin are discussed above.

Various dyes may be used to impart color to the cultivated mycelium material or composite mycelium material such as acid dyes, direct dyes, disperse dyes, sulfur dyes, synthetic dyes, reactive dyes, pigments (e.g. iron oxide black and cobalt blue) and natural dyes. In some embodiments, the cultivated mycelium material or composite mycelium material is submerged in an alkaline solution to facilitate dye uptake and penetration into the material prior to application of a dye solution. In some embodiments, the cultivated mycelium material or composite mycelium material is pre-soaked in ammonium chloride, ammonium hydroxide, and/or formic acid prior to application of a dye solution to facilitate dye uptake and penetration into the material. In some embodiments, tannins may be added to the dye solution. In various embodiments, the cultivated mycelium material or composite mycelium material may be preserved as discussed above before dye treatment or pre-treatment.

Depending on the embodiment, the dye solution may be applied to the cultivated mycelium material or composite mycelium material using different application techniques. In some embodiments, the dye solution may be applied to the one or more exterior surfaces of the cultivated mycelium material or composite mycelium material. In other embodiments, the cultivated mycelium material or composite mycelium material may be submerged in the dye solution.

In addition to pre-soaking with various solutions, agents may be added to the dye solution to facilitate dye uptake and penetration into the material. In some embodiments, ammonium hydroxide and/or formic acid with an acid or direct dye to facilitate dye uptake and penetration into the material. In some embodiments, an ethoxylated fatty amine is used to facilitate dye uptake and penetration into the processed material.

In various embodiments, a plasticization agent is added after or during the addition of the dye. In various embodiments, the plasticization agent may be added with the dye solution. In specific embodiments, the plasticization agent may be coconut oil, vegetable glycerol, or a sulfited or sulfated fatliquor.

In some embodiments, the dye solution may be maintained at a basic pH using a base such as ammonium hydroxide. In specific embodiments, the pH will be at least 9, 10, 11 or 12. In some embodiments, the pH of the dye solution will be adjusted to an acidic pH in order to fix the dye using various agents such as formic acid. In specific embodiments, the pH will be adjusted to a pH less than 6, 5, 4 or 3 in order to fix the dye.

In various methods, the cultivated mycelium material, composite mycelium material, and/or preserved composite mycelium material may be subject to mechanical working or agitation while the dye solution is being applied in order to facilitate dye uptake and penetration into the material. In some embodiments, subjecting the cultivated mycelium material, composite mycelium material, and/or preserved composite mycelium material to squeezing or other forms of pressure while in a dye solution enhanced dye uptake and penetration. In some embodiments, the cultivated mycelium material, composite mycelium material, and/or preserved composite mycelium material may be subject to sonication.

Using the methods described herein, the cultivated mycelium material or composite mycelium material may be dyed or colored such that the color of the processed cultivated mycelium material or composite mycelium material is substantially uniform. In some embodiments, the cultivated mycelium material or composite mycelium material is colored with the dye and the color of the cultivated mycelium material or composite mycelium material is substantially uniform on one or more surfaces of the cultivated mycelium material or composite mycelium material. Using the methods described above, the cultivated mycelium material or composite mycelium material may be dyed or colored such that dye and color is not just present in the surfaces of the cultivated mycelium material or composite mycelium material but instead penetrated through the surface to the inner core of the material. In such embodiments, the dye is present throughout the interior of the cultivated mycelium material or composite mycelium material.

In various embodiments of the present disclosure, the cultivated mycelium material or composite mycelium material may be dyed so that the cultivated mycelium material or composite mycelium material is colorfast. Colorfastness may be measured using various techniques such as ISO 11640:2012: Tests for Color Fastness—Tests for color fastness—Color fastness to cycles of to-and-fro rubbing or ISO 11640:2018 which is an update of ISO 11640:2012. In a specific embodiment, colorfastness will be measured according to the above using a Grey Scale Rating as a metric to determine rub fastness and change to sample. In some embodiments, the cultivated mycelium material or composite mycelium material will demonstrate strong colorfastness indicated by a Grey Scale Rating of at least 3, at least 4 or at least 5.

### Protein Sources

In various embodiments, it may be beneficial to optionally treat the cultivated mycelium material or composite mycelium material with one or more protein sources that are not naturally occurring in the cultivated mycelium material or composite mycelium material (i.e. exogenous protein sources). In some embodiments, the one or more proteins are from a species other than a fungal species from which the cultivated mycelium material is generated. In some embodiments, the cultivated mycelium material or composite mycelium material may be optionally treated with a plant protein source such as pea protein, rice protein, hemp protein and soy protein. In some embodiments, the protein source will be an animal protein such as an insect protein or a mammalian protein. In some embodiments, the protein will be a recombinant protein produced by a microorganism. In some embodiments, the protein will be a fibrous protein such as silk or collagen. In some embodiments, the protein will be an elastomeric protein such as elastin or resilin. In some embodiments, the protein will have one or more chitin-binding domains. Exemplary proteins with chitin-binding domains include resilin and various bacterial chitin-binding proteins. In some embodiments, the protein will be an engineered or fusion protein including one or more chitin-binding domains. Depending on the embodiment, the cultivated mycelium material or composite mycelium material may be preserved, as described above, before treatment or treated without prior preservation.

In a specific embodiment of the disclosure, the cultivated mycelium material or composite mycelium material is submerged in a solution including the protein source. In a specific embodiment, the solution including the protein source is aqueous. In other embodiments, the solution including the protein source includes a buffer such as a phosphate buffered saline.

In some embodiments, the solution including the protein source will include an agent that functions to crosslink the protein source. Depending on the embodiment, various known agents that interact with functional groups of amino acids can be used. In a specific embodiment, the agent that functions to crosslink the protein source is transglutaminase. Other suitable agents that crosslink amino acid functional groups include tyrosinases, genipin, sodium borate, and lactases. In other embodiments, traditional tanning agents may be used to crosslink proteins including chromium, vegetable tannins, tanning oils, epoxies, aldehydes and syntans. As discussed above, due to toxicity and environmental concerns with chromium, PAE other minerals may be used such as aluminum, titanium, zirconium, iron and combinations thereof with and without chromium.

In various embodiments, treatment with a protein source may occur before, after or concurrently with preserving the cultivated mycelium material or composite mycelium material, plasticizing the cultivated mycelium material or composite mycelium material and/or dyeing the cultivated mycelium material or composite mycelium material. In some embodiments, treatment with a protein source may occur before or during preservation of the cultivated mycelium material or composite mycelium material using a solution including alcohol and salt. In some embodiments, treatment with a protein source occurs before or concurrently with dyeing the cultivated mycelium material or composite mycelium material. In some of these embodiments, the protein source is dissolved in the dye solution. In a specific embodiment, the protein source will be dissolved in a basic dye solution optionally including one or more agents to facilitate dye uptake.

In some embodiments, a plasticizer will be added to the dye solution including the dissolved protein source to concurrently plasticize the cultivated mycelium material or composite mycelium material. In a specific embodiment, the plasticizer may be a fatliquor. In a specific embodiment, a plasticizer will be added to a protein source that is dissolved in a basic dye solution including one or more agents to facilitate dye uptake.

### Coating and Finishing Agents

After a cultivated mycelium material or composite mycelium material has been processed using any combination of methods as described above, the cultivated mycelium material or composite mycelium material may be treated with a finishing agent or coating. Various finishing agents common to the leather industry such as proteins in binder solutions, nitrocellulose, synthetic waxes, natural waxes, waxes with protein dispersions, oils, polyurethane, acrylic polymers, acrylic resins, emulsion polymers, water-resistant polymers and various combinations thereof may be used. In a specific embodiment, a finishing agent including nitrocellulose may be applied to the cultivated mycelium material or composite mycelium material. In another specific embodiment, a finishing agent including conventional polyurethane finish will be applied to the cultivated mycelium material or composite mycelium material. In various embodiments, one or more finishing agents will be applied to the cultivated mycelium material or composite mycelium material sequentially. In some instances, the finishing agents will be combined with a dye or pigment. In some instances, the finishing agents will be combined with a handle modifier (i.e. feel modifier or touch) including one or more of natural and synthetic waxes, silicone, paraffins, saponified fatty substances, amides of fatty acids, amides esters, stearic amides, emulsions thereof, and any combination of the foregoing. In some instances, the finishing agents will be combined with an antifoam agent. In some embodiments, an external element or force is applied to the cultivated mycelium material or composite mycelium material. In such embodiments, the external element or force includes heating and/or pressing. In some embodiments, the external element or force is hot pressing. In some embodiments, an external force is applied to the cultivated mycelium material or composite mycelium material. In such embodiments, the external force includes heating and/or pressing. In some embodiments, the external force is hot pressing.

### Processed Mycelium Material

In various embodiments of the present disclosure, the cultivated mycelium material or composite mycelium material is sonicated, perforated, or vacuum-processed. Perforation may include needle-punching, air-punching, or water-punching.

In various embodiments of the present disclosure, the cultivated mycelium material or composite mycelium material may be mechanically processed and/or chemically processed in different ways both in solution (i.e. dye solution, protein solution or plasticizer) and after the cultivated mycelium material or composite mycelium material has been removed from the solution. In such embodiments, the method includes mechanically processing and/or chemically processing the cultivated mycelium material or composite mycelium material, wherein a processed mycelium material is produced.

While the cultivated mycelium material or composite mycelium material is in a solution or dispersion it may be agitated, sonicated, squeezed or pressed to ensure uptake of the solution. The degree of mechanical processing will depend on the specific treatment being applied and the level of fragility of the cultivated mycelium material or composite mycelium material at its stage in processing. Squeezing or pressing of the cultivated mycelium material or composite mycelium material may be accomplished by hand wringing, mechanical wringing, a platen press, a lino roller or a calendar roller.

Similarly, as discussed above, the cultivated mycelium material or composite mycelium material may be pressed or otherwise worked to remove solution from the composite mycelium material after it is removed from solution. Treating with a solution and pressing the material may be repeated several times. In some embodiments, the material is pressed at least two times, at least three times, at least four times, or at least five times.

Once the cultivated mycelium material or composite mycelium material is fully dried (e.g. using heat, pressing or other desiccation techniques described above), the cultivated mycelium material or composite mycelium material may be subject to additional mechanical- and/or chemical-processing. Depending on the technique used to treat the cultivated mycelium material or composite mycelium material and the resultant toughness of the cultivated mycelium material or composite mycelium material, different types of mechanical processing may be applied including but not limited to sanding, brushing, plating, staking, tumbling, vibration and cross-rolling.

In some embodiments, the cultivated mycelium material or composite mycelium material may be embossed with any heat source or through the application of chemicals. In some embodiments, the cultivated mycelium material or composite mycelium material in solution may be subjected to additional chemical processing, such as, e.g., being maintained at a basic pH using a base such as ammonium hydroxide. In specific embodiments, the pH will be at least 9, 10, 11 or 12. In some embodiments, the pH of the cultivated mycelium material or composite mycelium material in solution will be adjusted to an acidic pH in order to fix the composite mycelium material using various agents such as formic acid. In specific embodiments, the pH will be adjusted to a pH less than 6, 5, 4 or 3 in order to fix the cultivated mycelium material or composite mycelium material.

Finishing, coating and other steps may be performed after or before mechanical processing and/or chemical processing of the dried cultivated mycelium material or composite mycelium material. Similarly, final pressing steps, including ornamental steps such as embossing or engraving, may be performed after or before mechanical processing and/or chemical processing of the dried cultivated mycelium material or composite mycelium material.

The raw mycelium material can be dried, refrigerated, or frozen material made according to any of the processes described herein. The raw material may optionally be split on the top and/or bottom to provide a mycelium panel having the desired thickness. Splitting can also provide a smoother surface at the cut. The crust material can be dyed, plasticized, dried and/or otherwise post-processed as described herein.

The pre-finishing treatment solution can include one or more dyes, tannins, and/or plasticizers (e.g. fatliquors) in a suitable solvent, such as water. In one example, the pre-finishing treatment solution includes one or more dyes and/or tannins and one or more fatliquors. The amount of dye added can be based on the particular type of dye and the desired color of the resulting product. An exemplary pre-finishing treatment solution includes: one or more acid dyes at a concentration to produce the desired color; about 25 g/L vegetable tannins; about 6.25 g/L Truposol® LEX fatliquour (Trumpler, Germany); and about 18 g/L to about 19 g/L Trupon® DXV fatliquor (Trumpler, Germany).

The pre-finishing treatment solution can be applied to the mycelium material through a combination of soaking and pressing processes. In one example, the material is soaked in the pre-finishing treatment solution for a predetermined period of time (e.g., 1 minute) and then moved through a pressing system. An example of a suitable pressing system includes moving the soaked material through a pair of rollers that are spaced to provide the desired degree of pressing to the material with each pass between the rollers. The material can be pushed and/or pulled through the rollers. The rate at which the material is passed through the rollers can vary. According to one aspect of the present disclosure, the soaking and pressing process can be repeated one or more times (e.g., 1, 2, 3, 4, 5 or more times).

Following the pre-finishing treatment application, the material can proceed to a fixation process. The fixation process includes adjusting the pH of the pre-finishing treatment solution to a pH suitable for fixing the dyes. In one example, the fixation process is an acid fixing process that includes decreasing the pH of the pre-finishing treatment solution. Non-limiting examples of acids suitable for acid fixing include acetic acid and formic acid. For example, acetic acid can be used to decrease the pH of the exemplary pre-finishing treatment solution described above to a pH of 3.15±1.0.

The mycelium material can be soaked in the pH adjusted pre-finishing treatment solution and flattened in a manner similar to that described above. The soaking and pressing process can be repeated one or more times (e.g., 1, 2, 3, 4, 5 or more times).

A final, extended soak of the material in the pH adjusted pre-finishing treatment solution can be done. The material can be inverted about halfway through the extended soak period. The extended soak period can be from about 30 minutes to 1 hour or more. When the extended soak time period is complete, the material can be processed through a final pressing process. The final pressing process can be the same or different than that described above.

Following the fixation process, the material can be dried with or without heating. The material can be held generally vertically, horizontally, or any orientation therebetween during the drying step. The material may optionally be restrained during the drying step. For example, one or more clamps may be used to restrain all or a portion of the material during drying. In some examples, the drying step 216 is conducted at ambient conditions.

### Mechanical Properties of Composite Mycelium Material

Various methods of the present disclosure may be combined to provide processed cultivated or composite mycelium material that has a variety of mechanical properties. In such embodiments, the mycelium material includes a mechanical property, e.g., a wet tensile strength, an initial modulus, an elongation percentage at the break, a thickness, and/or a slit tear strength. Other mechanical properties include, but are not limited to, elasticity, stiffness, yield strength, ultimate tensile strength, ductility, hardness, toughness, creep resistance, and other mechanical properties known in the art.

In various embodiments, the processed mycelium material may have a thickness that is less than 1 inch, less than ½ inch, less than ¼ inch or less than ⅛ inch. In some embodiments, the composite mycelium material has a thickness of about 0.5 mm to about 3.5 mm, inclusive, e.g., about 0.5 mm to about 1.5 mm, about 1 mm to about 2.5 mm, and about 1.5 mm to about 3.5 mm. The thickness of the material within a given piece of material may have varying coefficients of variance. In some embodiments, the thickness is substantially uniform to produce a minimal coefficient of variance.

In some embodiments, the mycelium material can have an initial modulus of at least 20 MPa, at least 25 MPa, at least 30 MPa, at least 40 MPa, at least 50 MPa, at least 60 MPa, at least 70 MPa, at least 80 MPa, at least 90 MPa, at least 100 MPa, at least 110 MPa, at least 120 MPa, at least 150 MPa, at least 175 MPa, at least 200 MPa, at least 225 MPa, at least 250 MPa, at least 275 MPa, at least 300 MPa, at least 325 MPa, at least 350 MPa, at least 375 MPa, at least 400 MPa, or at least 500 MPa. In some embodiments, the mycelium material may have an initial modulus of about 0.5 MPa to about 500 MPa, inclusive, for example about 0.5 MPa to about 10 MPa, about 1 MPa to about 20 MPa, about 10 MPa to about 30 MPa, about 20 MPa to about 40 MPa, about 30 MPa to about 50 MPa, about 40 MPa to about 60 MPa, about 50 MPa to about 70 MPa, about 60 MPa to about 80 MPa, about 70 MPa to about 90 MPa, about 80 MPa to about 100 MPa, about 90 MPa to about 150 MPa, about 100 MPa to about 200 MPa, about 150 MPa to about 300 MPa, about 200 MPa to about 300 MPa, about 300 MPa to about 400 MPa, about 350 MPa to about 500 MPa, and about 40 MPa to about 500 MPa. In specific embodiments, the mycelium material has an initial modulus of 0.8 MPa. In one aspect, the mycelium material has an initial modulus of 1.6 MPa. In another aspect, the mycelium material has an initial modulus of 97 MPa.

In some embodiments, the mycelium material can have a wet tensile strength of about 0.05 MPa to about 50 MPa, inclusive, e.g., about 1 MPa to about 5 MPa, about 5 MPa to about 20 MPa, about 10 MPa to about 30 MPa, about 15 MPa to about 40 MPa, and about 20 MPa to about 50 MPa. In specific embodiments, the mycelium material may have a wet tensile strength of about 5 MPa to about 20 MPa. In one aspect, the mycelium material has a wet tensile strength of about 7 MPa. In a specific embodiment, the wet tensile strength will be measured by ASTM D638.

In some embodiments, the mycelium material can have a breaking strength (“ultimate tensile strength”) of at least 1.1 MPa, at least 6.25 MPa, at least 10 MPa, at least 12 MPa, at least 15 MPa, at least 20 MPa, at least 25 MPa, at least 30 MPa, at least 35 MPa, at least 40 MPa, at least 45 MPa, at least 50 MPa.

In some embodiments, the mycelium material has an elongation at the break of less than 2%, less than 3%, less than 5%, less than 20%, less than 25%, less than 50%, less than 77.6%, or less than 200%. For example, the mycelium material may have an elongation at the break of about 1% to about 200%, inclusive, e.g., about 1% to about 25%, about 10% to about 50%, about 20% to about 75%, about 30% to about 100%, about 40% to about 125%, about 50% to about 150%, about 60% to about 175%, and about 70% to about 200%.

In some embodiments, the initial modulus, ultimate tensile strength, and elongation at the break are measured using ASTM D2209 or ASTM D638. In a specific embodiment, the initial modulus, ultimate tensile strength, and elongation at the break are measured using a modified version ASTM D638 that uses the same sample dimension as ASTM D638 with the strain rate of ASTM D2209.

In some embodiments, the mycelium material can have a single stitch tear strength of at least 15N, at least 20N, at least 25N, at least 30N, at least 35N, at least 40N, at least 50N, at least 60N, at least 70N, at least 80N, at least 90N, at least 100N, at least 125N, at least 150N, at least 175N, or at least 200N. In a specific embodiment, the tongue tear strength will be measured by ASTM D4786.

In some embodiments, the mycelium material can have a double stitch tear strength of at least 20N, at least 40N, at least 60N, at least 80N, at least 100N, at least 120N, at least 140N, at least 160N, at least 180N, or at least 200N. In a specific embodiment, the tongue tear strength will be measured by ASTM D4705.

In some embodiments, the mycelium material can have a tongue tear strength (also referred to as slit tear strength) of at least 1.8N, at least 15N, at least 25N, at least 35N, at least 50N, at least 75N, at least 100N, at least 150N, or at least 200N, as measured by ISO-3377. In a specific embodiment, the tongue tear strength will be measured by ASTM D4704. In some embodiments, the mycelium material may have a slit tear strength of at least 1N, at least 20N, at least 40N, at least 60N, at least 80N, at least 100N, at least 120N, at least 140N, at least 160N, at least 180N, or at least 200N, as measured by ISO-3377-2. In one aspect, the mycelium material has a slit tear strength of about 1N to about 200N, inclusive, e.g., about 10N to about 30N, about 20N to about 40N, about 30N to about 50N, about 40N to about 60N, about 50N to about 70N, about 60N to about 80N, about 70N to about 90N, about 80N to about 100N, about 90N to about 110N, about 100N to about 120N, about 110N to about 130N, about 120N to about 140N, about 130N to about 150N, about 140N to about 160N, about 150N to about 170N, about 160N to about 180N, about 170N to about 190N, and about 180N to about 200N, as measured by ISO-3377-2.

In some embodiments, the mycelium material has a flexural modulus (Flexure) of at least 0.2 MPa, at least 1 MPa, at least 5 MPa, at least 20 MPa, at least 30 MPa, at least 50 MPa, at least 80 MPa, at least 100 MPa, at least 120 MPa, at least 140 MPa, at least 160 MPa, at least 200 MPa, at least 250 MPa, at least 300 MPa, at least 350 MPa, at least 380 MPa. In a specific embodiment, the compression will be measured by ASTM D695. In some embodiments, the mycelium material has a flexural modulus (Flexure) of less than 0.2 MPa, less than 1 MPa, less than 5 MPa, less than 20 MPa, less than 30 MPa, less than 40 MPa, less than 50 MPa, less than 60 MPa, less than 70 MPa, less than 80 MPa, less than 90 MPa, less than 100 MPa, less than 110 MPa, less than 120 MPa, less than 130 MPa, less than 140 MPa, less than 150 MPa, less than 160 MPa, less than 200 MPa, less than 250 MPa, less than 300 MPa, less than 350 MPa, less than 380 MPa. In some embodiments, the mycelium material has a flexural modulus of about 5-10 MPa. In some embodiments, the mycelium material has a flexural modulus of about 10-15 MPa. In some embodiments, the mycelium material has a flexural modulus of about 10-20 MPa. In some embodiments, the mycelium material has a flexural modulus of about 20-30 MPa. In some embodiments, the mycelium material has a flexural modulus of about 30-40 MPa. In some embodiments, the mycelium material has a flexural modulus of about 40-50 MPa. In some embodiments, the mycelium material has a flexural modulus of about 50-60 MPa. In some embodiments, the mycelium material has a flexural modulus of about 60-70 MPa. In some embodiments, the mycelium material has a flexural modulus of about 70-80 MPa. In some embodiments, the mycelium material has a flexural modulus of about 10-11 MPa. In some embodiments, the mycelium material has a flexural modulus of about 10 MPa. In some embodiments, the mycelium material has a flexural modulus of about 20 MPa. In some embodiments, the mycelium material has a flexural modulus of about 30 MPa. In some embodiments, the mycelium material has a flexural modulus of about 40 MPa. In some embodiments, the mycelium material has a flexural modulus of about 50 MPa. In some embodiments, the mycelium material has a flexural modulus of about 60 MPa. In some embodiments, the mycelium material has a flexural modulus of about 70 MPa. In some embodiments, the mycelium material has a flexural modulus of about 80 MPa. In some embodiments, the mycelium material has a flexural modulus of about 90 MPa. In some embodiments, the mycelium material has a flexural modulus of about 100 MPa. In a specific embodiment, the compression will be measured by ASTM D695.

In various embodiments of the present disclosure, the mycelium material has different absorption properties measured as a percentage mass increase after soaking in water. In some embodiments, the percent mass increase after soaking in water for 1 hour is less than 1%, less than 5%, less than 25%, less than 50%, less than 74%, or less than 92%. In a specific embodiment, the percent mass increase after soaking in water after 1 hour is measured using ASTM D6015.

### Methods of Producing a Mycelium Material

Also provided is a method of producing a mycelium material as described herein. According to one embodiment of the disclosure, a mycelium material can be produced by generating a cultivated mycelium material including one or more masses of branching hyphae; disrupting the cultivated mycelium material including the one or more masses of branching hyphae; and adding a bonding agent to the cultivated mycelium material (e.g., by contacting the disrupted cultivated mycelium material with a solution comprising a bonding agent); thus producing the composite mycelium material. In some embodiments, the cultivated mycelium material includes one or more masses of disrupted branching hyphae. In some embodiments, the one or more masses of disrupted branching hyphae has a length. In such embodiments, the one or more masses of disrupted branching hyphae has a length of about 0.1 mm to about 5 mm.

In another aspect, a mycelium material ca be produced by generating a cultivated mycelium material; pressing the cultivated mycelium material; and adding a bonding agent to the cultivated mycelium material (e.g., by contacting the pressed cultivated mycelium material with a solution comprising a bonding agent), thus producing the composite mycelium material.

In some embodiments, the generating comprises generating cultivated mycelium material on a solid substrate. In some embodiments, the method further comprises incorporating a supporting material into the mycelium material. In some embodiments, the supporting material is a reinforcing material. In some embodiments, the supporting material is a base material. In some embodiments, the disrupting comprises disrupting the one or more masses of branching hyphae by a mechanical action. In some embodiments, the method further comprises adding one or more proteins that are from a species other than a fungal species from which the cultivated mycelium material is generated. In some embodiments, the method further comprises adding a dye to the cultivated mycelium material or the mycelium material. In some embodiments, the method further comprises adding a plasticizer to the cultivated mycelium material or the mycelium material. In some embodiments, the method further comprises adding a tannin to the cultivated mycelium material or the mycelium material. In some embodiments, the method further comprises adding a finishing agent to the mycelium material. In some embodiments, the method further comprises determining a mechanical property of the mycelium material, wherein the mechanical property includes, but is not limited to, wet tensile strength, initial modulus, elongation percentage at the break, thickness, slit tear strength, elasticity, stiffness, yield strength, ultimate tensile strength, ductility, hardness, toughness, creep resistance, and the like. For example, the mycelium material has a wet tensile strength of about 0.05 MPa to about 50 MPa, an initial modulus of about 0.5 MPa to about 300 MPa, an elongation percentage at the break of about 1% to about 200%, a thickness of about 0.5 mm to about 3.5 mm, and/or a slit tear strength of about 1 N to about 200 N.

In some embodiments, the cultivated mycelium material or composite mycelium material is produced using traditional paper milling equipment.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); and the like.

### Example 1: OSA as Lubricant in Mycelium Material

15 g of mycelium biomass was added to 1 L water and blended in a Blendtec at 1.5%, at setting 5, for 90 seconds. The resulting slurry was sieved with a 500 mesh sieve to remove any liquid. The mycelia was then re-dispersed in the Blendtec at 1 for 10 s in 1 L water. Four slurries were prepared. One slurry was stirred at room temperature (RT) without any lubricant or binding agent as a control. 12.8 mL 2-octenyl succinic anhydride (OSA, 1 mole equivalent to the polysaccharide amount of the mycelium biomass) was slowly added with stirring at RT to the remaining 3 slurries. 0.5 mL 10M NaOH was added to all three slurries to initiate the reaction between the anhydride from OSA and the hydroxyl groups from the glucans in the hyphae polysaccharide. The final pH was 8.5. The slurries were stirred at room temperature for 4 hours. All four slurries were vacuum filtered to remove unreacted chemicals and re-dispersed in 1 L water. Different amounts of Dur-o-Set Elite Plus binder was added to two of the slurries before wet laying, either 5 g or 9.8 g. The four slurries were then wet-laid onto a Buchner vacuum flask to form a web. All four webs were dried at 45° C., then pressed at 90° C. and 20 kN for 2 minutes and conditioned in the chamber before testing. Table 1 provides a summary of the production method of the four cultivated mycelium panels.

TABLE 1

| | Slurry | | | |
|---|---|---|---|---|
| Sample | Conc (wt %) | Lubricant | Binder | pH |
| 1 | 1.5 | — | — | |
| 2 | 1.5 | 12.8 g OSA + NaOH | — | 8.5 |
| 3 | 1.5 | 12.8 g OSA + NaOH | 5 g Elite Plus | 8.5 |
| 4 | 1.5 | 12.8 g OSA + NaOH | 9.8 g | 8.5 |
| | | | Elite Plus | |

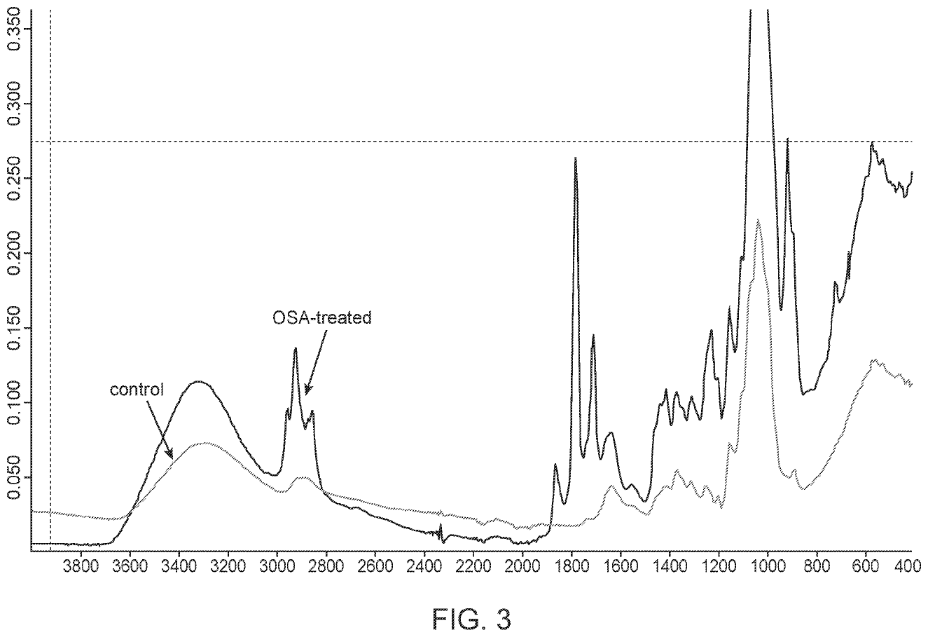

Incorporation of the OSA into the treated material was determined by ATR-FTIR. ATR-FTIR spectrum of control mycelium and OSA treated material was collected from 4000 cm⁻¹ to 400 cm⁻¹ which showed additional peaks in the aliphatic region after OSA treatment. As shown in FIG. 3, OSA was incorporated into the material.

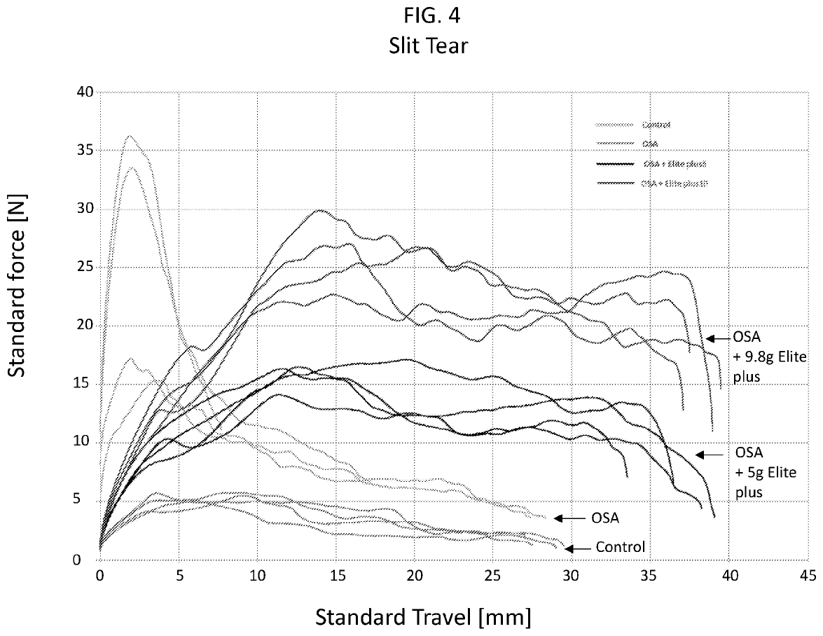

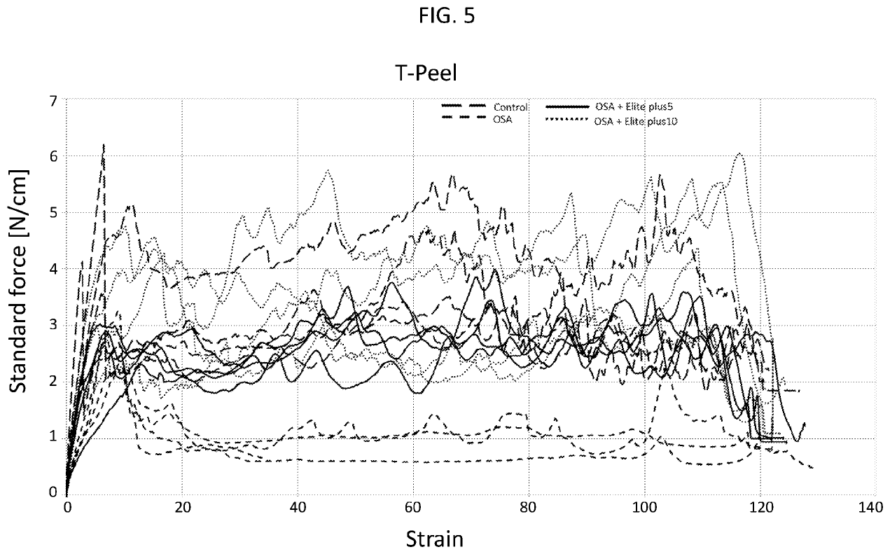

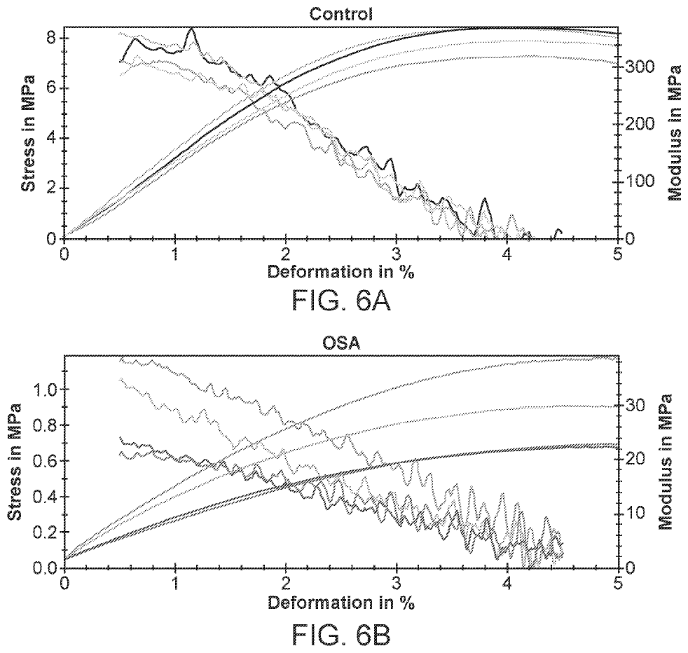

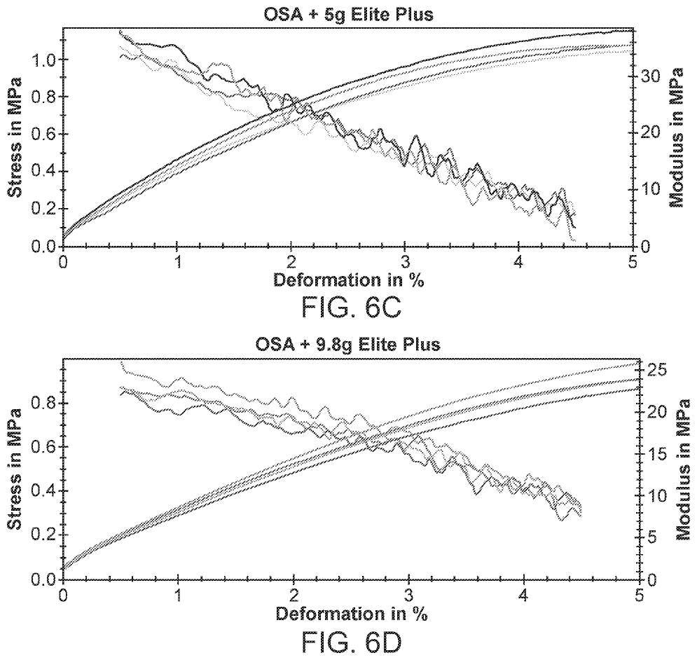

The various mechanical properties of the mycelium material after the various properties were assessed. Slit tear was determined by ISO 3377-2. This test measured the force required to rupture a pre slit material. Samples were conditioned at 65±2% RH for 24 hours. In some embodiments, samples were equilibrated at 65% relative humidity for 16 h at room temperature prior to testing. The ISO 3377-2 die was used to cut out 1″×2″ specimens with a center slit. The appropriate slit tear test method was then run on the universal mechanical tester from Zwick. T-peel was determined by broadly following ASTM D1876 using rubber mycelium bonding. Peel strength determined the interlaminar resistance of the material. In brief, a notch was cut in the z-direction of the material such that two layers formed by the notch had comparable thickness on each side. The force required for complete delamination of the material was measured as peel force. Flexural modulus was determined by the industry standard test ASTM D790-03. In brief, the sample was deflected until the outer surface ruptures or until a maximum strain of 5.0% was reached, whichever occurs first. The procedure employs a strain rate of 0.01 mm/mm/min. The slit tear results are shown in FIG. 4. The T-peel results are shown in FIG. 5. The flexural modulus results are shown in FIG. 6A-D.

A summary of the control and treated mycelium materials' properties is provided in

Table 2.

TABLE 2

| | Vol | Vol | | | | | | | |
|---|---|---|---|---|---|---|---|---|---|
| | Density | Density | | | | | | Flexural | Flexural |
| | before | after | | | | | | Modulus | Modulus |
| | pressing | pressing | | Thickness | Slit Tear | T-peel F | T-Peel F | Max | Avg |
| Sample | (g/cm3) | (g/cm3) | GSM | (mm) | (N) | Max | Avg | (MPa) | (MPa) |
| 1 | 0.28 | 0.58 | 675 | 1.16 ± 0.03 | 26 ± 11 | 5.17 ± 0.9 | 3.13 ± 0.68 | 340 ± 27 | 219 ± 19 |
| Control | | | | | | | | | |
| 2 OSA | 0.36 | 0.85 | 980 | 1.15 ± 0.01 | 5.5 ± 0.27 | 2.7 ± 0.47 | 1.03 ± 0.18 | 30 ± 8 | 18 ± 5.3 |
| 3 OSA + | 0.61 | 0.91 | 1151 | 1.26 ± 0.02 | 16 ± 1.3 | 3.46 ± 0.48 | 2.52 ± 0.2 | 37 ± 2 | 22 ± 0.8 |
| 5 g | | | | | | | | | |
| Elite | | | | | | | | | |
| Plus | | | | | | | | | |
| 4 OSA + | 0.53 | 0.78 | 1236 | 1.59 ± 0.16 | 27 ± 2 | 5.27 ± 0.72 | 3.35 ± 0.75 | 24 ± 2 | 17.5 ± 0.9 |
| 9.8 g | | | | | | | | | |
| Elite | | | | | | | | | |
| Plus | | | | | | | | | |

Volumetric densities were tracked before and after pressing for all the samples. The volumetric density increases by ˜0.3 g/cm3 after pressing in Carver hot press for all samples improving the overall aesthetic feel and fullness of all the samples.

The inclusion of OSA during the wet lay process significantly decreased the flexural modulus of the material as compared to untreated control material. Functionalization with OSA reduced the flexural modulus of mycelium from 219N to 18N. OSA functionalized webs were flexible even without any fatliquor. The addition of the binder did not affect the flexibility of the OSA treated material.

Control mycelium without any additives shows high initial slit tear strength (26N) but overall tear propagation strength remained low. Functionalization of hyphae with OSA lowered the slit tear to 5.5N with constant tear propagation strength observed throughout the tear.

Similar behavior was observed in the T-peel strength where the functionalization with OSA reduces the initial max T-peel and overall average T-peel strength, possibly as a result of lubrication which disrupted the internal hydrogen bonding within the hyphae.

Addition of Elite plus improved the slit tear strength of the OSA functionalized samples irrespective of thickness. Elite plus also improved the overall tear propagation behavior with higher Elite plus showing higher slit tear strength. The slit tear force increased from 5.5 N to 16 N or 27 N, depending on the amount of binder added, and the T peel average increased from 1.03 to 2.52 or 3.35, depending on the amount of binder added. Thus, the addition of OSA significantly increased the flexibility of the mycelium material, and the ability of the material to withstand tearing or peeling forces can be improved by the addition of a binder without adversely affecting the flexibility of the OSA-treated material.

OSA has been used to modify starches, but the ability of OSA to provide internal lubrication and impart significant flexibility on a fibrous material (e.g., the mycelium material) was not expected.

### Example 2: Siloxanes as Lubricant in Mycelium Material

15 g mycelium biomass was added to 1 L water and blended in the Blendtec at 1.5%, at setting 5, for 90 seconds. The resulting slurry was sieved with a 500 mesh sieve to remove water soluble components of the mycelium. Starsoft siloxane was added to the slurries so that the final concentration in the final product was 8-15 wt % siloxane. 8 g of Elite Plus binder (50 wt % solids) was also added to the slurry. Two such slurries were prepared with final StarSoft concentrations of 10% and 13%. The mycelia were then re-dispersed in the Blendtec at 1 for 10 s in 1 L water. The slurries were then wet-laid via vacuum filtration in a 6 inch Buchner funnel on a forming cloth. Four webs were made, two with siloxane, and two control webs. All four webs were dried at 45° C., then pressed twice at 90° C., at 20 kN for 2 minutes with a 0.8 mm shim and then a 0.65 mm shim and conditioned in a humidity chamber at 50% RH and at 21° C. before testing. One control web was left untreated and the other was treated with 10% DXV/LEX fat liquor. Bovine leather and mango fruit leather were used as controls as well. The flexural modulus of the samples was assessed as described in Example 1.

The flexural modulus results of the siloxane-treated mycelium materials and leather controls are summarized in Table 3 below.

TABLE 3
Table 3. Flexure modulus comparison of mycelium material with leather samples.

| | | | Flexure Modulus | |
|---|---|---|---|---|
| Samples | Description | n = ? | (MPa) | Std Dev (MPa) |
| PN2052 | mycelium with DXV/LEX | 3 | 69 | 16 |
| | Fat liquor, 10% in web | | | |
| CR1262 | mycelium with no lubricant | 3 | 289 | 5 |
| CR1263 | mycelium with 10% | 3 | 126 | 21 |
| | StarSoft-Bis-45 | | | |
| CR1264 | mycelium with 13% | 3 | 65 | 7 |
| | StarSoft-Bis-45 | | | |
| Bovine Leather | | 3 | 2 | 1 |
| Mango Fruitleather | | 3 | 22 | 3 |

The addition of fat liquor or siloxane significantly reduced the flexural modulus of the mycelium material compared to material with no treatment. In addition, the samples treated with 13% siloxane had a similar flexural module as the samples treated with the traditional fat liquor finishing agent. Thus, the addition of siloxane at an early stage of the material production resulted in increased flexibility of the material.

In addition, the amount of siloxane used in the treatment can be altered to achieve a desired material flexibility. For instance, for stiffer material, less siloxane can be used, while more siloxane can be used to produce a more flexible material.

Siloxanes have been used as finishing agents in the textile industry, but not as beginning products, as used here. Without intending to be bound by theory, the addition of the siloxane at the end stage of the mycelium material synthesis process is likely to not be as successful at imparting flexibility, smoothness, and drape as the addition of the siloxane during the initial mycelium material synthesis process. This is because the siloxane may not permeate through a finished fibrous product as evenly or efficiently, whereas the addition of the siloxane prior to material drying, such as at the blending or wet-laying step, allows for the even and thorough incorporation of the siloxane throughout the material. If a siloxane is used during early production, the fat liquor step later in production may be removed if desired.

It will be understood by one having ordinary skill in the art that construction of the described disclosure and other components is not limited to any specific material. Other exemplary embodiments of the disclosure disclosed herein may be formed from a wide variety of materials, unless described otherwise herein.

It is also important to note that the construction and arrangement of the elements of the disclosure as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. Accordingly, all such modifications are intended to be included within the scope of the present disclosure. Other substitutions, modifications, changes, and omissions may be made in the design, operating conditions, and arrangement of the desired and other exemplary embodiments without departing from the spirit of the present disclosure.

It will be understood that any described processes or steps within processes described herein may be combined with other disclosed processes or steps to form structures within the scope of the present disclosure. The exemplary structures and processes disclosed herein are for illustrative purposes and are not to be construed as limiting.

1. A composite mycelium material, comprising a cultivated mycelium material comprising one or more masses of branching hyphae, and a siloxane.

2. The composite mycelium material of claim 1, wherein the siloxane comprises a hydroxysilicone, a silicone hydride, an epoxy silicone, an aminosilicone, or an alkyl ethylene oxide condensate.

3. The composite mycelium material of claim 1 or 2, wherein the composite mycelium material comprising a siloxane has a lower flexural modulus as compared to a cultivated mycelium material alone.

4. A composite mycelium material, comprising a cultivated mycelium material comprising one or more masses of branching hyphae, and an aliphatic chain compound covalently linked to the one or more masses of branching hyphae.

5. The composite mycelium material of claim 4, wherein the aliphatic chain compound comprises 2-octenyl succinic anhydride (OSA), 2-dodecenyl succinic anhydride, octadecenyl succinic anhydride, stearic anhydride, 3-Chloro-2-hydroxypropyldimethyldodecylammonium chloride, heptanoic anhydride, butyric anhydride, or a chlorohydrin.

6. The composite mycelium material of claim 4 or 5, wherein the composite mycelium material comprising an aliphatic chain compound has a lower flexural modulus as compared to a cultivated mycelium material alone.

7. The composite mycelium material of any one of claims 1-6, wherein the one or more masses of branching hyphae is disrupted.

8. The composite mycelium material of any one of claims 1-6, wherein the cultivated mycelium material is pressed.

9. The composite mycelium material of any one of claims 1-8, wherein the composite mycelium material has a flexural modulus of less than 80 MPa.

10. The composite mycelium material of any one of claims 1-8, wherein the composite mycelium material has a flexural modulus of 1 MPa to 80 MPa.

11. The composite mycelium material of any one of claims 1-8, wherein the composite mycelium material has a flexural modulus of at least about 1, 5, 10, 15, 20, 25, 30, 35, 40, 45, 50, 55, 60, 65, 70, 75, or 80 MPa.

12. The composite mycelium material of any one of claims 1-11, wherein the composite mycelium material is more flexible as compared to a cultivated mycelium material alone.

13. The composite mycelium material of any one of claims 1-12, wherein the composite mycelium material further comprises a bonding agent.

14. The composite mycelium material of claim 13, wherein the bonding agent comprises one or more reactive groups.

15. The composite mycelium material of claim 14, wherein the one or more reactive groups react with active hydrogen containing groups.

16. The composite mycelium material of claim 15, wherein the active hydrogen containing groups comprise amine, hydroxyl, and carboxyl groups.

17. The composite mycelium material of any one of claims 13-16, wherein the bonding agent comprises an adhesive, a resin, a crosslinking agent, and/or a matrix.

18. The composite mycelium material of any one of claims 13-17, wherein the bonding agent is selected from the group consisting of a vinyl acetate-ethylene (VAE) copolymer, a vinyl acetate-acrylic copolymer, a polyamide-epichlorohydrin resin (PAE), a copolymer, transglutaminase, citric acid, genipin, alginate, gum arabic, latex, a natural adhesive, and a synthetic adhesive.

19. The composite mycelium material of claim 18, wherein the bonding agent is a copolymer with a property selected from the group consisting of: a particle size of less than or equal to 1 μm, a sub-zero glass transition temperature, and self-crosslinking function.

20. The composite mycelium material of claim 18 or 19, wherein the bonding agent is a vinyl acetate-ethylene (VAE) copolymer.

21. The composite mycelium material of any one of claims 1-20, wherein the composite mycelium material further comprises a dye.

22. The composite mycelium material of claim 21, wherein the dye is selected from the group consisting of an acid dye, a direct dye, a synthetic dye, a natural dye, and a reactive dye.

23. The composite mycelium material of claim 21 or 22, wherein the composite mycelium material is colored with the dye and the color of the composite mycelium material is substantially uniform on one or more surfaces of the composite mycelium material.

24. The composite mycelium material of any one of claims 21-23, wherein the dye is present throughout the interior of the composite mycelium material.

25. The composite mycelium material of any one of claims 1-24, wherein the composite mycelium material further comprises a plasticizer.

26. The composite mycelium material of claim 25, wherein the plasticizer is selected from the group consisting of oil, glycerin, fatliquor, sorbitol, diethyloxyester dimethyl ammonium chloride, Tween 20, Tween 80, m-erythritol, water, glycol, triethyl citrate, water, acetylated monoglycerides, and epoxidized soybean oil.

27. The composite mycelium material of any one of claims 1-26, wherein the composite mycelium material further comprises a tannin.

28. The composite mycelium material of any one of claims 1-27, wherein the composite mycelium material further comprises a finishing agent.

29. The composite mycelium material of claim 28, wherein the finishing agent is selected from the group consisting of urethane, wax, nitrocellulose, and a plasticizer.

30. The composite mycelium material of any one of claims 1-29, wherein the cultivated mycelium material has been generated on a solid substrate.

31. The composite mycelium material of any one of claims 1-30, wherein the one or more masses of branching hyphae are entangled, wherein the entangling the hyphae comprises hydroentangling, needle punching or felting.

32. The composite mycelium material of any one of claims 1-30, wherein the one or more masses of branching hyphae is disrupted by a mechanical action.

33. The composite mycelium material of claim 32, wherein the mechanical action comprises blending the one or more masses of branching hyphae.

34. The composite mycelium material of any one of claims 1-33, wherein the mechanical property comprises a wet tensile strength, an initial modulus, an elongation percentage at the break, a thickness, and/or a slit tear strength.

35. A method of producing a composite mycelium material, the method comprising:
a. generating a cultivated mycelium material comprising one or more masses of branching hyphae; and
b. adding a siloxane to the cultivated mycelium material; thus producing the composite mycelium material.

36. The method of claim 35, further comprising disrupting or pressing the cultivated mycelium material generated in step (a).

37. The method of claim 36, wherein the siloxane is added before the masses of branching hyphae are disrupted, during disruption of the masses of branching hyphae, or after the disruption of the masses of branching hyphae.

38. The method of claim 36, wherein the siloxane is added before the pressing step, during the pressing step, or after the pressing step.

39. The method of claims 35-38, wherein the siloxane comprises a hydroxysilicone, a silicone hydride, an epoxy silicone, an aminosilicone, or an alkyl ethylene oxide condensate.

40. The method of claims 35-39, wherein the cultivated mycelium material comprising a siloxane has a lower flexural modulus as compared to a cultivated mycelium material without a siloxane.

41. A method of producing a composite mycelium material, the method comprising:
a. generating a cultivated mycelium material comprising one or more masses of branching hyphae; and
b. adding an aliphatic chain compound to the cultivated mycelium material; thus producing the composite mycelium material.

42. The method of claim 41, further comprising disrupting or pressing the cultivated mycelium material generated in step (a).

43. The method of claim 41, wherein the aliphatic chain compound is added before the masses of branching hyphae are disrupted, during disruption of the masses of branching hyphae, or after the disruption of the masses of branching hyphae.

44. The method of claim 41, wherein the aliphatic chain compound is added before the pressing step, during the pressing step, or after the pressing step.

45. The method of claims 41-44, wherein the aliphatic chain compound comprises 2-octenyl succinic anhydride (OSA), 2-dodecenyl succinic anhydride, octadecenyl succinic anhydride, stearic anhydride, 3-Chloro-2-hydroxypropyldimethyldodecylammonium chloride, heptanoic anhydride, butyric anhydride, or a chlorohydrin.

46. The method of any one of claims 41-45, wherein the cultivated mycelium material comprising an aliphatic chain compound has a lower flexural modulus as compared to a cultivated mycelium material without an aliphatic chain compound.

47. The method of any one of claims 35-46, wherein the composite mycelium material has a flexural modulus of less than 80 MPa.

48. The method of any one of claims 35-47, wherein the composite mycelium material has a flexural modulus of 1 MPA to 80 MPa.

49. The method of any one of claims 35-48, wherein the composite mycelium material has a flexural modulus of at least about 1, 5, 10, 15, 20, 25, 30, 35, 40, 45, 50, 55, 60, 65, 70, 75, or 80 MPa.

50. The method of any one of claims 35-49, wherein the composite mycelium material is more flexible as compared to a cultivated mycelium material alone.

51. The method of any one of claims 35-50, wherein composite mycelium material further comprises a bonding agent.

52. The method of claim 51, wherein the bonding agent comprises one or more reactive groups.

53. The method of claim 52, wherein the one or more reactive groups react with active hydrogen containing groups.

54. The method of claim 53, wherein the active hydrogen containing groups comprise amine, hydroxyl, and carboxyl groups.

55. The method of any one of claims 51-54, wherein the bonding agent comprises an adhesive, a resin, a crosslinking agent, and/or a matrix.

56. The method of any one of claims 51-55, wherein the bonding agent is selected from the group consisting of a vinyl acetate-ethylene (VAE) copolymer, a vinyl acetate-acrylic copolymer, a polyamide-epichlorohydrin resin (PAE), a copolymer, transglutaminase, citric acid, genipin, alginate, gum arabic, latex, a natural adhesive, and a synthetic adhesive.

57. The method of claim 56, wherein the bonding agent is a copolymer with a property selected from the group consisting of: a particle size of less than or equal to 1 μm, a sub-zero glass transition temperature, and self-crosslinking function.

58. The method of claim 56 or 57, wherein the bonding agent is a vinyl acetate-ethylene (VAE) copolymer.

59. The method of any one of claims 35-58, wherein the composite mycelium material further comprises a dye.

60. The method of claim 59, wherein the dye is selected from the group consisting of an acid dye, a direct dye, a synthetic dye, a natural dye, and a reactive dye.

61. The method of claim 59 or 60, wherein the composite mycelium material is colored with the dye and the color of the composite mycelium material is substantially uniform on one or more surfaces of the composite mycelium material.

62. The method of any one of claims 59-61, wherein the dye is present throughout the interior of the composite mycelium material.

63. The method of any one of claims 35-62, wherein the composite mycelium material further comprises a plasticizer.

64. The method of claim 63, wherein the plasticizer is selected from the group consisting of oil, glycerin, fatliquor, sorbitol, diethyloxyester dimethyl ammonium chloride, Tween 20, Tween 80, m-erythritol, water, glycol, triethyl citrate, water, acetylated monoglycerides, and epoxidized soybean oil.

65. The method of any one of claims 35-64, wherein the composite mycelium material further comprises a tannin.

66. The method of any one of claims 35-65, wherein the composite mycelium material further comprises a finishing agent.

67. The method of claim 66, wherein the finishing agent is selected from the group consisting of urethane, wax, nitrocellulose, and a plasticizer.

68. The method of any one of claims 35-67, wherein the cultivated mycelium material has been generated on a solid substrate.

69. The method of any one of claims 35-68 further comprising entangling the one or more masses of branching hyphae, wherein the entangling the hyphae comprises hydroentangling, needle punching, or felting.

70. The method of any one of claims 35-69, wherein the disrupting comprises disrupting the one or more masses of branching hyphae by a mechanical action.

71. The method of claim 70, wherein the mechanical action comprises blending the one or more masses of branching hyphae.
